(19) United States
(12) Patent Application Publication
(10) Pub. No.: US 2026/0290384 A1
(43) Pub. Date: Sep. 24, 2026

(54) SYSTEM

(71) Applicant: SoftBank Group Corp., Tokyo (JP)

(72) Inventor: Satoshi WADA, Tokyo (JP)

(73) Assignee: SoftBank Group Corp., Tokyo (JP)

(21) Appl. No.: 19/568,885

(22) Filed: Mar. 17, 2026

(30) Foreign Application Priority Data
Mar. 19, 2025 (JP) 2025-045271

(51) Int. Cl. G10L 25/66; G10L 15/26; G10L 25/30; G10L 25/63; G16H 50/30

(52) U.S. Cl. CPC G10L 25/66; G10L 15/26; G10L 25/30; G10L 25/63; G16H 50/30

(57) ABSTRACT

A system comprising a processor, wherein the processor is configured to: record voice of a user through a mobile device and convert voice data into text data by using a speech recognition technique, perform emotion analysis on the converted text data by using a natural language processing technique and assign emotion tags to the converted text data, and generate a prompt for instructing a generative AI model to analyze signs of dementia, based on notified information.

### CROSS-REFERENCE TO RELATED APPLICATION

This application is based on and claims priority under 35 USC 119 from Japanese Patent Application No. 2025-045271 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional dementia screening techniques primarily rely on periodical medical examinations or questionnaires administered in clinical settings. As a result, early and subtle signs of cognitive decline in daily life are frequently overlooked, and continuous monitoring of a user's mental state in a natural environment is difficult to achieve. Moreover, existing systems that record user speech or behavior often stop at simple logging or basic statistical analysis, and do not fully utilize advanced natural language processing or generative AI models to interpret emotional states or complex behavioral patterns that may indicate dementia. In addition, even when suspicious patterns appear in the user's speech, there is often no timely mechanism to automatically notify family members or care managers, causing delays in appropriate medical consultation or intervention. Therefore, there is a need for a system that can continuously acquire speech data through a mobile device, convert the speech data into text, perform emotion analysis on the text, and automatically generate prompts for a generative AI model to analyze signs of dementia, and that can further detect behavioral patterns suspected of dementia and notify related parties when predetermined criteria are exceeded.

### SUMMARY

In order to solve the above problems, the present invention provides a system comprising a processor, wherein the processor is configured to record voice of a user through a mobile device and convert voice data into text data by using a speech recognition technique, perform emotion analysis on the converted text data by using a natural language processing technique and assign emotion tags to the converted text data, and generate a prompt for instructing a generative AI model to analyze signs of dementia, based on notified information. The processor is further configured to analyze stored data to detect a behavioral pattern suspected of dementia, and to notify a related party when a detected behavioral pattern exceeds a predetermined criterion. By integrating continuous speech acquisition via the mobile device, emotion tagging of the converted text data, prompt generation for the generative AI model, detection of dementia-suspected behavioral patterns from stored data, and automatic notification to related parties, the system enables early identification of dementia signs and timely communication to caregivers and other stakeholders.

The term “processor” refers to one or more hardware computing elements, such as a central processing unit (CPU), graphics processing unit (GPU), digital signal processor (DSP), application-specific integrated circuit (ASIC), or programmable logic device, that execute instructions to perform the functions described in the system.

The term “mobile device” refers to a portable electronic device, such as a smartphone, tablet, wearable device, or other handheld terminal, that includes at least a microphone, a processor, and a communication interface, and that is capable of recording user voice and transmitting data to the system.

The term “voice data” refers to digital audio data representing sound captured from the user's speech by a microphone of the mobile device, including raw or encoded waveforms suitable for speech recognition processing.

The term “text data” refers to a sequence of characters or tokens obtained by converting voice data into written language using a speech recognition technique.

The term “speech recognition technique” refers to a software or hardware implemented method, including but not limited to automatic speech recognition (ASR) algorithms or models, that receives voice data as input and outputs corresponding text data.

The term “natural language processing technique” refers to a software or hardware implemented method for analyzing and processing human language in text form, including but not limited to tokenization, parsing, sentiment analysis, and emotion classification.

The term “emotion analysis” refers to processing that evaluates text data to determine one or more emotional states expressed or implied in the text, such as happiness, sadness, anger, fear, confusion, anxiety, or neutrality.

The term “emotion tag” refers to a label or data element associated with text data that indicates an estimated emotional state or sentiment, and that may include a categorical label, a numerical score, or a combination thereof.

The term “notified information” refers to information provided to or obtained by the system, including at least the emotion tags, analysis results, or other metadata derived from the user's speech or behavior, which serve as a basis for generating a prompt for a generative AI model.

The term “prompt” refers to a text or structured input that is generated by the processor and supplied to a generative AI model to instruct the model to perform a specific analysis or task, including analysis of signs of dementia.

The term “generative AI model” refers to a machine learning model, such as a large language model or similar generative model, that generates outputs including text, scores, or structured information in response to a prompt, and that is used to analyze signs of dementia based on the prompt and associated data.

The term “signs of dementia” refers to linguistic, emotional, or behavioral indicators that may suggest cognitive impairment consistent with dementia, including but not limited to frequent forgetfulness, disorientation, repetition of questions, or abnormal emotional patterns observed in the user's speech.

The term “stored data” refers to data saved in a storage device or database of the system, including text data converted from voice data, emotion tags, prompts, analysis results, behavioral records, and associated timestamps and metadata.

The term “behavioral pattern” refers to a recurring or characteristic combination of user behaviors or utterances over time, derived from stored data, that reflects trends or tendencies in the user's speech, emotions, or interactions.

The term “behavioral pattern suspected of dementia” refers to a behavioral pattern that, according to predetermined rules, models, or thresholds, is evaluated as likely to be associated with dementia or early cognitive decline.

The term “predetermined criterion” refers to a reference value, rule set, model threshold, or condition defined in advance, against which one or more features of a detected behavioral pattern are compared in order to determine whether an alert or notification should be generated.

The term “related party” refers to a person or entity associated with the user's care or monitoring, including but not limited to a family member, caregiver, care manager, healthcare professional, or other designated contact to whom the system sends notifications.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

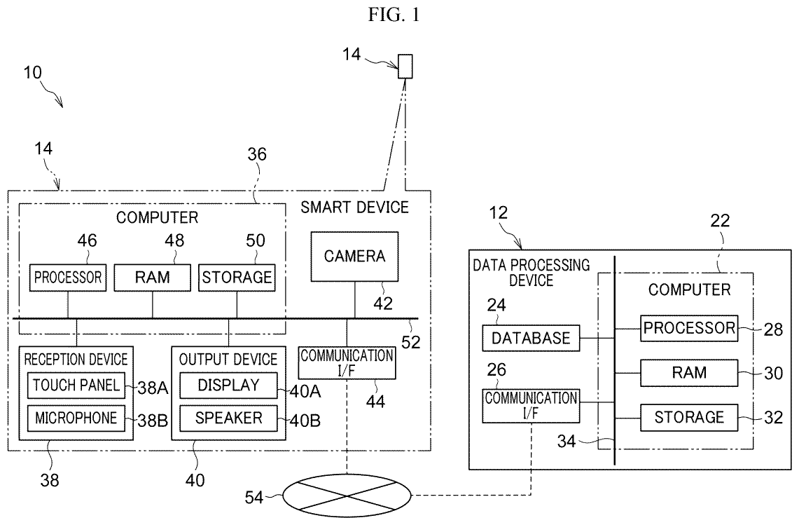

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

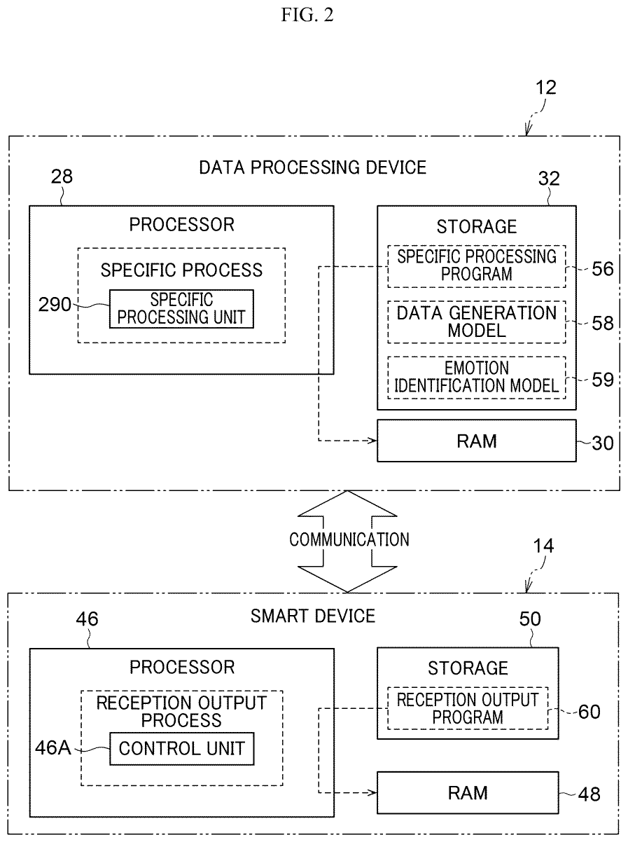

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented monitoring systems that aim to detect cognitive decline from user behavior typically suffer from several technical limitations. First, such systems often treat speech-to-text conversion, sentiment analysis, data storage, and pattern detection as loosely coupled components, without an integrated feedback mechanism that optimizes the overall processing pipeline. As a result, errors or bias in upstream components, such as emotion analysis, propagate downstream into pattern detection models, leading to reduced detection accuracy and instability over time. This causes inefficient utilization of computing resources and requires frequent manual tuning by specialists.

Second, known systems generally rely on static machine learning models and fixed feature sets defined at design time. When user behavior, language usage, or environmental conditions change, these static configurations become suboptimal. Updating them typically requires human experts to redesign features, adjust thresholds, and reconfigure notification conditions, which is difficult to scale, particularly in distributed mobile and server environments. Consequently, the systems cannot adapt in a timely manner to new data distributions, and their performance degrades.

Third, existing architectures lack a technically integrated mechanism to leverage generative AI models within the core analytics loop. While generative AI models can, in principle, suggest improved feature engineering strategies, analysis policies, and threshold settings, conventional systems do not define a structured way to generate prompt sentences based on concrete statistical and model outputs, nor do they define how to systematically incorporate the generative AI outputs back into the operational sentiment analysis and behavioral pattern detection processes. This results in ad hoc, manual use of generative AI, rather than a repeatable, machine-executable improvement cycle.

Fourth, in many known solutions, the data flow from mobile acquisition to server-side analysis is not optimized for cumulative, time-series oriented analysis of emotion-annotated text data. Systems may record text or emotions independently, or only at coarse granularity, without generating feature information that captures temporal changes, label distributions, phrase frequencies, and model output transitions. This leads to inefficient database queries and weak support for time-based behavioral trend analysis, which are important for early detection of cognitive dysfunction.

Accordingly, there is a need for a computer-implemented system that improves the technical operation of cognitive impairment detection by: (i) tightly integrating voice acquisition, speech recognition, emotion-annotated text generation, structured data storage, and model-based pattern detection; (ii) automatically generating analysis features and behavioral patterns suitable for machine learning; and (iii) using a generative AI model, driven by automatically formulated prompt sentences derived from accumulated statistical and model outputs, to adaptively refine the sentiment analysis and behavioral pattern detection processes. Such a system should reduce manual intervention, improve detection accuracy and robustness over time, and enhance the efficiency and scalability of the underlying computing infrastructure.

The specific processing by the specific processing unit 290 of the data processing device 12 in Example 1 is realized by the following means.

The present invention provides a server comprising a processor and a storage device storing instructions that, when executed by the processor, cause the server to (i) receive from a portable terminal device voice-derived character information and associated user and time information, execute emotion analysis processing on the character information using a natural language processing program to produce emotion-annotated character information including emotion labels and emotion scores, and store the emotion-annotated character information in a data management storage device in a structured manner; (ii) generate, from sets of the stored emotion-annotated character information, feature information representing behavioral tendencies suspected of being related to cognitive dysfunction, construct a machine learning model, including a classification or discrimination model, that estimates a presence or degree of suspicion of cognitive dysfunction based on the feature information, and execute behavioral pattern detection for each user using the machine learning model; and (iii) compute statistical information over time for the emotion labels, emotion scores, phrase occurrences, and model outputs, automatically generate a prompt sentence describing at least one of an analysis policy, a feature configuration, a threshold configuration, and a notification configuration for cognitive dysfunction detection based on the behavioral pattern detection results and the statistical information, transmit the prompt sentence to a generative information processing model, receive response information from the generative information processing model, and, in accordance with the response information, automatically update at least one of parameters, processing content, or configuration of the emotion analysis processing and the behavioral pattern detection processing. This enables an integrated, computer-implemented feedback loop in which the server continuously refines sentiment analysis and pattern detection pipelines based on accumulated time-series data and generative AI guidance, thereby improving detection accuracy, robustness to changing data distributions, and computational efficiency without requiring frequent manual reconfiguration.

The term “system” refers to an information processing arrangement including at least one processor and at least one storage device, optionally distributed across one or more server devices and terminal devices, that cooperatively execute programmed instructions to perform the operations described in the claims.

The term “processor” refers to a hardware computation unit, such as a central processing unit, a microprocessor, or an equivalent processing circuit, capable of executing machine-readable instructions to control input, output, storage, and analysis operations.

The term “portable terminal device” refers to a user-operated electronic apparatus that is physically portable and includes at least an input unit, an output unit, a processor, a memory, and a communication interface, such as a smartphone, a tablet device, or a handheld terminal.

The term “acoustic input unit” refers to an input component that captures sound waves and converts the sound waves into electrical or digital signals, including but not limited to a microphone, an audio sensor, and associated audio interface circuitry.

The term “operation control application” refers to an executable software program or software module installed on the portable terminal device and configured to control acquisition, buffering, and transmission of voice signals and related data in response to user actions or predefined conditions.

The term “voice signal” refers to an analog or digital representation of speech produced by a user, including raw audio data captured by the acoustic input unit and any processed version of the audio data suitable for speech recognition.

The term “speech recognition processing unit” refers to a hardware and/or software component configured to receive voice signals and convert the voice signals into character information using a speech recognition algorithm, which may include acoustic modeling, language modeling, and decoding processing.

The term “character information” refers to text-based digital data representing a transcription of speech, including characters, words, or sentences encoded in a machine-readable format.

The term “natural language processing program” refers to a software component or library configured to perform computational analysis of character information, including at least tokenization, syntactic or semantic analysis, and determination of emotion-related attributes.

The term “emotion analysis processing” refers to a sequence of computational operations that analyze character information to determine emotional characteristics, including at least one of polarity, intensity, and category of emotion, based on linguistic features or statistical models.

The term “emotion-annotated character information” refers to character information to which one or more emotion labels and optionally one or more emotion scores are attached as metadata to represent emotional characteristics of the underlying text.

The term “emotion label” refers to a categorical indicator associated with character information that represents an emotional state, such as a positive state, a negative state, a neutral state, or another predefined emotion category.

The term “emotion score” refers to a numerical value or vector of numerical values associated with character information, representing a quantitative measure of emotional intensity, polarity, or related attributes.

The term “user identification information” refers to data that identifies or distinguishes one user from another, such as a user ID, account identifier, pseudonymous token, or other identifier managed by the system.

The term “time information” refers to temporal data associated with an event or data record, such as a timestamp, date, time interval, or period indicator, representing when the voice signal was acquired or when the character information was generated or processed.

The term “data management storage device” refers to a memory or storage subsystem, including one or more physical or virtual storage resources, such as databases, file systems, or storage arrays, configured to store, retrieve, and manage structured or unstructured data.

The term “feature information” refers to data derived from one or more items of emotion-annotated character information and related metadata, formatted as numerical or symbolic features suitable for input to a machine learning model.

The term “behavioral tendency” refers to a pattern or trend in user behavior over time inferred from multiple data points, such as repeated emotional states, recurring phrases, or consistent changes in linguistic or temporal characteristics.

The term “cognitive dysfunction” refers to an impairment or abnormality in cognitive functions, such as memory, attention, judgment, or reasoning, which may indicate or be associated with a neurological or psychological disorder.

The term “machine learning processing program” refers to a software component or set of components configured to execute machine learning algorithms, including at least data preprocessing, model training, inference, and evaluation, using feature information as input.

The term “classification model” refers to a machine learning model configured to assign input feature information to one of a plurality of predefined categories, such as categories representing presence or absence of suspected cognitive dysfunction.

The term “discrimination model” refers to a machine learning model configured to distinguish between at least two classes or states, such as a normal cognitive state and a suspected cognitive dysfunction state, and to output an estimate or score representing class membership or likelihood.

The term “behavioral pattern” refers to a set of one or more behavioral tendencies or feature combinations, identified by analysis of stored data, that is indicative of or correlated with a particular state, such as a sign of cognitive dysfunction.

The term “statistical information” refers to aggregate data computed from one or more records, including but not limited to counts, frequencies, distributions, averages, variances, or temporal trends of labels, scores, or other metrics.

The term “analysis policy” refers to a set of rules, criteria, or procedures that govern how data is analyzed, including which features are used, how data is segmented, how thresholds are applied, and how detection results are interpreted or classified.

The term “feature design” refers to a specification or configuration defining how raw data and metadata are transformed into feature information, including selection, extraction, transformation, and combination of variables used as inputs to a machine learning model.

The term “threshold setting” refers to a configuration of one or more numerical or categorical boundary values used to decide between different outcomes, such as whether a degree of suspicion of cognitive dysfunction is high enough to trigger a particular action.

The term “notification condition setting” refers to a configuration defining the conditions under which the system initiates a notification, including criteria based on model outputs, thresholds, time windows, or user-specific preferences.

The term “prompt sentence” refers to a textual input sequence constructed by the system and supplied to a generative information processing model, the textual input describing a context, a question, a request for suggestions, or other content to elicit a desired response.

The term “generative information processing model” refers to a machine learning model configured to generate text or structured information in response to an input prompt sentence, based on learned patterns from training data, such as a generative AI model.

The term “response information” refers to data output by the generative information processing model in response to a prompt sentence, including natural language text, configuration suggestions, feature recommendations, or other machine-usable content.

The term “processing content” refers to a specification of which operations are executed and in what manner within a processing pipeline, including selection of algorithms, processing stages, and data flow sequences.

The term “parameter” refers to a configurable value or set of values controlling the behavior of a processing operation or model, such as thresholds, weights, learning rates, or configuration options used in emotion analysis or behavioral pattern detection.

The term “notification process” refers to a sequence of computational operations in which the system determines that a notification condition is satisfied, constructs notification data, and transmits the notification data to one or more recipient devices or services.

The term “degree of suspicion of the cognitive dysfunction” refers to a quantitative or qualitative estimate output by the classification model or the discrimination model that represents the likelihood or severity of suspected cognitive dysfunction for a user.

The term “electronic communication network” refers to a wired or wireless communication infrastructure, such as the Internet, a local area network, or a mobile communication network, over which digital data can be transmitted between devices.

The term “guardian” refers to a person or entity responsible for overseeing the welfare of the user, such as a family member, caregiver, or legal representative, as designated in the system.

The term “healthcare professional” refers to an individual engaged in providing medical or clinical care, such as a physician, psychologist, nurse, or therapist, who may receive notifications or reports from the system.

The term “alert information” refers to a message, signal, or data payload that indicates to a recipient that a particular condition of interest, such as a high degree of suspicion of cognitive dysfunction, has been detected by the system.

The term “time-series data” refers to data elements associated with time information, ordered or analyzed according to temporal sequence, enabling evaluation of changes and trends over time.

In one embodiment, a server cooperates with a terminal to implement a computer-implemented system for detecting signs of cognitive dysfunction from speech-based user interaction. The server comprises at least one processor, at least one main memory, a non-transitory storage device, and a network interface. The terminal comprises at least one processor, a memory, a high-sensitivity microphone as an acoustic input unit, an audio codec, a display unit, and a wireless communication interface. The server and the terminal execute software modules including an operation control application on the terminal, a speech recognition client, a natural language processing module, a machine learning analysis module, and an interface module to a generative AI model.

The terminal executes an operation control application running on a mobile operating system, such as a smartphone operating system or a tablet operating system. The terminal controls an audio driver and an audio codec to convert an analog voice signal generated by a user into digital audio samples. The terminal uses an audio capture application programming interface, such as a platform audio recording interface, to buffer and packetize the audio samples into a digital audio file with a predetermined sampling rate, bit depth, and encoding format. By specifying a compact yet sufficient sampling format, the terminal reduces communication load while maintaining adequate recognition accuracy for downstream speech recognition.

The terminal transmits the audio file to a speech recognition processing unit. In one embodiment, the terminal uses an application programming interface of a remote speech recognition service accessed over a secure communication protocol. The terminal generates a request message including the audio data and metadata such as language code and sampling rate, and sends the request through a transport layer protocol stack. The speech recognition processing unit executes acoustic modeling, feature extraction such as Mel-frequency cepstral coefficient computation, and decoding using a statistical or neural language model. The speech recognition processing unit returns character information in a text-based format together with confidence scores for recognized segments.

The server, in another embodiment, performs the speech recognition processing locally. The server executes a speech recognition engine that includes a neural network-based acoustic model and a decoding graph stored in memory. The server loads the audio file transmitted from the terminal, performs framing, windowing, and feature extraction, and applies the acoustic model to generate posterior probabilities over phonetic units. The server then executes a Viterbi or beam-search decoder to output a best path of word sequences as character information. The server stores the character information in association with user identification information and time information.

The server executes a natural language processing program to perform emotion analysis processing on the character information. The server loads a linguistic processing library and initializes a tokenizer, part-of-speech tagger, and sentiment classification module. The server segments the character information into tokens and sentences, assigns part-of-speech tags, and maps tokens to feature indices. The server constructs feature vectors comprising, for example, term frequencies, n-gram counts, syntactic dependency flags, and lexicon-based emotion scores. The server applies a sentiment classification model, which in one embodiment is a logistic regression classifier or a gradient boosting classifier trained on annotated corpora, to output an emotion label and an emotion score. The server associates the resulting label (for example, positive, negative, neutral) and the score (for example, a continuous value from −1.0 to +1.0) with the original character information to generate emotion-annotated character information.

The server stores the emotion-annotated character information in a data management storage device. The server defines a structured data schema, for example including a user identifier field, a timestamp field, a text field, an emotion label field, and an emotion score field. The server writes each unit of emotion-annotated character information as a record into a relational table or into a logically equivalent structured data store. By indexing the user identifier and timestamp fields, the server allows efficient retrieval of time-series emotion data for each user, thereby improving query speed for downstream behavioral analysis.

The server aggregates emotion-annotated character information for each user to generate feature information representing behavioral tendencies. The server computes statistics such as moving averages of emotion scores over predefined windows, frequencies of particular emotion labels, counts of specific phrases associated with forgetfulness or confusion, and temporal gradients of sentiment change. The server forms feature vectors that combine these statistics with additional indicators such as time-of-day usage patterns. In one embodiment, the server uses a sliding window mechanism and precomputed cumulative sums to derive such window-based features, thereby reducing computational overhead compared to recomputing statistics from raw records.

The server constructs a machine learning model based on the feature information. The server selects an algorithm structure, such as a random forest classifier or a support vector machine, and initializes model parameters. During training, the server splits the available feature information into training and validation sets, computes a loss function such as cross-entropy loss, and updates model parameters via an optimization procedure. In another embodiment, the server constructs a neural network model with multiple fully connected layers, rectified linear unit activation functions, and a softmax output layer. The server initializes layer weights and applies a backpropagation algorithm with a gradient-based optimizer to minimize the loss function. The server may apply regularization methods, such as dropout or L2 regularization, to prevent overfitting. The server stores the trained model parameters as part of a reusable model object in persistent storage.

The server executes behavioral pattern detection by applying the trained model to newly generated feature information. The server inputs a feature vector representing recent behavior of a user into the model and obtains an output representing a degree of suspicion of cognitive dysfunction. The server interprets the output, which may be a probability value or a confidence score, and compares it to a configurable threshold. When the output exceeds the threshold, the server identifies a behavioral pattern indicating a sign of cognitive dysfunction for that user. Because the server uses derived feature information that reflects temporal emotion dynamics and phrase usage, rather than single utterances in isolation, the behavioral pattern detection is more robust and less susceptible to transient anomalies.

The server performs statistical processing on emotion-annotated character information and model outputs to produce statistical information. The server computes distributions of emotion labels over extended time periods, histograms of emotion scores, occurrence frequencies of terms related to memory or confusion, and trends in the model's output scores. The server stores these statistical summaries in a separate data structure, such as a summary table or a multidimensional time-series array. This separation between raw records and aggregated summaries enables the server to respond to analytical queries with reduced latency and lower input/output overhead, resulting in improved computational efficiency.

The server automatically generates a prompt sentence based on detection results and statistical information. The server formulates the prompt sentence by inserting numerical values and qualitative descriptors into a predefined template. For example, the server may generate a prompt sentence such as: “The system records user conversations via a mobile terminal, converts speech to text, and assigns emotion tags with a natural language processing module. We want to detect early cognitive impairment using a machine learning model on aggregated sentiment and language features. Which additional features, sentiment analysis strategies, or model architectures should we consider to improve sensitivity while limiting false positives?”

In another example, the server may generate a prompt sentence such as:
“Over the last 30 days, the proportion of negative emotion labels for a user has increased from 10% to 45%, and references to forgetfulness-related terms have doubled. The current classification model uses only aggregate sentiment scores and label frequencies. Propose a feature design and threshold setting that can better distinguish normal mood variation from early cognitive impairment.”

The server transmits the prompt sentence to a generative AI model through a network interface. The generative AI model, in one embodiment, is a neural network with a transformer architecture including multiple self-attention layers, feedforward layers, and positional encodings. The server sends the prompt sentence as tokenized text input and receives response information in the form of generated text describing recommended feature engineering approaches, analysis policies, or threshold configurations. The server parses the response information to extract machine-usable parameters, such as a list of suggested features, recommended ranges for threshold values, or a model architecture description.

The server updates configuration of its sentiment analysis and behavioral pattern detection processing based on the response information. The server may, for instance, add new features that measure lexical diversity, changes in average utterance length, or variability of sentiment within a conversation session. The server may adjust the threshold for suspicion scores or modify the balance between sensitivity and specificity by altering the decision rule. The server writes updated configuration settings into a configuration store and refreshes the corresponding software modules without requiring complete manual redesign. Because the server uses structured internal representations of features and thresholds, the incorporation of generative AI suggestions is not a trivial automation of human decisions, but a technical mechanism to reconfigure internal data processing pipelines and models in a principled and repeatable manner.

The server, upon detecting that the degree of suspicion of cognitive dysfunction exceeds a predetermined criterion, executes a notification process. The server retrieves contact information for a guardian, a healthcare professional, or the user from the data management storage device, constructs an alert message including summary information about detected behavioral patterns, and transmits the alert message over an electronic communication network. The server may use standardized communication protocols to send messages to terminal devices, such as push notifications or email messages. The server may also update a monitoring dashboard accessible to authorized users, presenting visualization of emotion trends and risk levels. These operations involve control of communication devices and data display units, thereby linking the processing to physical-world devices.

The terminal displays feedback to the user. The terminal presents simplified indicators of cognitive status or recommendations for follow-up actions, if configured. The terminal may prompt the user to answer additional questions or perform cognitive exercises, the responses to which are processed by the server as additional input data. The feedback loop between user interaction, data acquisition, server analysis, and generative AI-driven reconfiguration allows the system to adapt to individual user profiles and changing patterns.

The described architecture improves computer technology in several ways. The server reduces processing latency and storage overhead by defining specific data structures for emotion-annotated character information and feature information, by using indexed storage, and by computing time-windowed statistics with incremental updates. This leads to faster execution compared to generic log-based analysis. The server increases detection accuracy by modeling temporal patterns of emotion and phrase usage, rather than relying on static, single-time-point observations. The server improves computational efficiency by decoupling raw data storage from aggregated representations and by using model-based selection of relevant features. The server reduces communication bandwidth requirements by performing audio compression and selective uploading, and by transmitting compact feature vectors rather than raw audio, in certain embodiments.

In contrast to conventional manual or rule-based monitoring, the server employs a non-conventional feedback mechanism integrating a generative AI model. The server does not merely automate human decision-making; rather, the server automatically generates prompt sentences grounded in quantitative system metrics and uses the generative AI outputs to adjust internal model parameters and processing flows. This changes the operation of the computer system itself by adapting feature extraction, model configurations, and threshold logic in response to observed data distributions and performance outcomes. The result is improved robustness against concept drift and reduced need for manual retraining or reconfiguration.

Alternative embodiments are possible. In one variation, the server executes the sentiment analysis module on the terminal to reduce server load, with the terminal transmitting only emotion-annotated character information to the server. In another variation, the server employs a deep neural network classifier that jointly ingests raw character sequences and emotion labels to learn composite patterns indicative of cognitive dysfunction, with an architecture that includes an embedding layer, convolutional layers to capture local n-gram patterns, and recurrent or attention-based layers to capture long-range dependencies. In an additional variation, the server uses active learning: the server selects ambiguous cases based on classification uncertainty and includes them in a training set with human-verified labels; the server then retrains the model with adjusted loss weighting to focus on previously misclassified patterns.

The server may also implement different optimization strategies, such as mini-batch gradient descent with adaptive learning rate schedules, different loss functions such as focal loss to handle class imbalance, and data augmentation strategies including paraphrasing or synonym substitution applied to text data. By explicitly specifying these technical processing steps, data structures, model architectures, and optimization techniques, the system operates as a concrete technological solution that enhances computational performance and accuracy in the detection of cognitive dysfunction, rather than as an abstract idea or mere automation of human analysis.

Through these configurations and variations, the server and the terminal collaboratively implement the claimed system in a manner that can be realized by persons skilled in the art, and that provides technical advantages in processing speed, detection precision, resource utilization, and adaptability of the computer system itself.

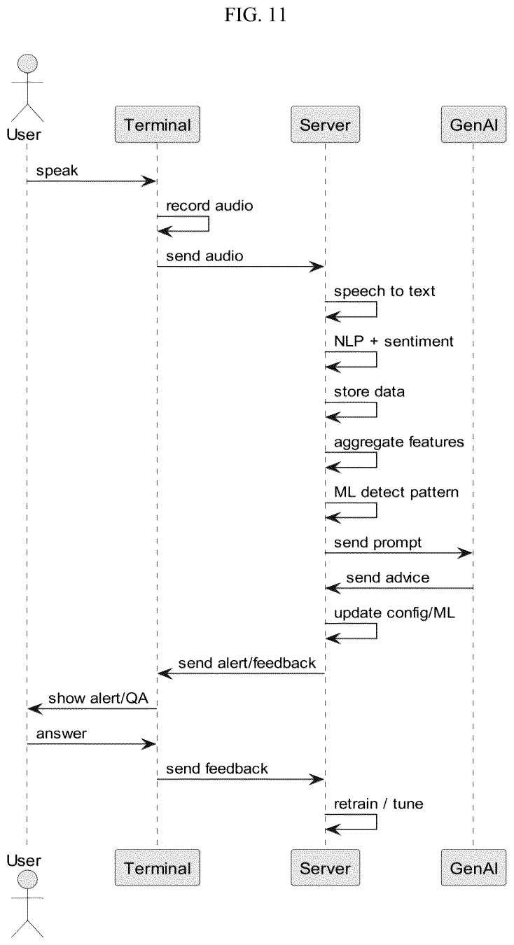

The following describes the processing flow using FIG. 11.

### Step 1

User provides speech.

User speaks naturally near the terminal about daily events, feelings, or difficulties, such as memory problems. The input is an analog voice signal produced by the user's vocal utterance. No digital output is produced at this step; instead, the acoustic signal is delivered to the terminal's microphone for further processing.

### Step 2

Terminal captures and digitizes audio.

Terminal activates an operation control application and controls its acoustic input unit and audio driver to start recording when a trigger condition is met (for example, app launch or scheduled monitoring). The input is the analog voice signal from the user. Terminal uses an audio codec to convert the analog signal into digital PCM samples with a specified sampling rate and bit depth. Terminal buffers the samples in memory and writes them into an audio file (for example, a WAV file) using an operating system audio recording interface. The output is a digital audio file stored in the terminal's local storage, associated with a temporary identifier and timestamp.

### Step 3

Terminal prepares and sends audio for speech recognition.

Terminal reads the stored audio file, adds metadata such as language code and sampling configuration, and forms a request payload for a speech recognition processing unit. The input is the digital audio file and associated metadata. Terminal may compress or re-encode the audio to reduce size, then transmits it over a secure communication channel to either a remote recognition service or a server-local recognition module. The output is a request message containing the audio data, delivered to the speech recognition processing unit.

### Step 4

Server or speech recognition unit converts audio to character information.

Server receives the request and passes the audio data to a speech recognition engine. The input is the digital audio signal from the terminal. Server segments the audio into frames, computes acoustic features such as Mel-frequency cepstral coefficients, and applies an acoustic model (for example, a neural network) to estimate phonetic probabilities. Server then uses a decoding algorithm, such as beam search with a language model, to select the most likely sequence of words. The output is character information, including a text transcription and optional confidence scores, which the server or speech recognition unit returns to the terminal or stores locally.

### Step 5

Server normalizes and stores raw character information.

Server receives the text transcription and associated metadata. The input is the character information with confidence scores, user identification information, and time information. Server normalizes the text by applying operations such as lowercasing, unifying punctuation, and removing non-speech artifacts according to predefined rules. Server then creates a structured record including user ID, timestamp, and normalized text, and stores this record in a data management storage device. The output is a stored text record that is ready for emotion analysis.

### Step 6

Server performs natural language preprocessing.

Server retrieves a text record from the storage device for analysis. The input is the normalized character information. Server executes a natural language processing program to tokenize the text into words and sentences, and applies part-of-speech tagging to each token. Server may also compute additional linguistic features, such as lemma forms, n-gram sequences, or dependency tags. The output is a structured representation of the text containing tokens, tags, and auxiliary linguistic attributes.

### Step 7

Server performs emotion analysis and generates emotion-annotated character information.

Server uses the structured text representation as input to an emotion analysis module. The input is the list of tokens, part-of-speech tags, and associated features. Server calculates feature vectors that may include term frequencies, n-gram presence indicators, lexicon lookups for emotionally charged words, and syntactic pattern flags. Server then applies a trained sentiment classification model, such as a logistic regression classifier or a neural network, to these feature vectors. The model computes an emotion score, for example a real-valued sentiment intensity, and predicts an emotion label, such as positive, negative, or neutral. The output is emotion-annotated character information, which contains the original text, the emotion label, and the emotion score.

### Step 8

Server stores emotion-annotated character information.

Server constructs a database record from the emotion-annotated character information. The input is the annotation result, including user ID, timestamp, text, emotion label, and emotion score. Server writes these fields into a table of the data management storage device and updates any relevant indexes for fast querying. The output is a persistent emotion-annotated record that can be accessed for time-series and behavioral analysis.

### Step 9

Server aggregates time-series emotion and phrase features.

Server periodically queries the data management storage device for emotion-annotated records belonging to each user within a given time window. The input is a set of stored emotion-annotated records retrieved by user ID and timestamp range. Server computes aggregate statistics, such as the count and ratio of each emotion label, average and variance of emotion scores, frequency of specific terms related to memory or confusion, and trends in sentiment over time. Server uses numerical operations, including summation, averaging, and differencing, to generate feature values. The output is feature information, represented as a feature vector per user per time window.

### Step 10

Server applies a machine learning model to detect behavioral patterns.

Server loads a trained classification or discrimination model from storage. The input is the feature vector describing behavioral tendencies for a user. Server feeds this vector into the model, which executes internal computations such as matrix multiplications and activation functions to produce an output score or probability that indicates a degree of suspicion of cognitive dysfunction. Server compares this score to one or more predefined thresholds. The output is a behavioral pattern detection result that flags whether the user is suspected of exhibiting signs of cognitive dysfunction and includes the associated suspicion score.

### Step 11

Server updates statistical summaries and model output history.

Server aggregates detection results over time for each user. The input is a sequence of behavioral pattern detection results and their timestamps. Server computes statistics such as the frequency of above-threshold suspicion events, rolling averages of suspicion scores, and distributions of model outputs across time intervals. Server inserts or updates summary records in a separate database structure. The output is a set of updated statistical summaries that reflect how the model's decisions and user behavior evolve over time.

### Step 12

Server generates a prompt sentence for a generative AI model.

Server uses the detection results and statistical summaries as input to a prompt generation module. The input is the statistical information, such as increased negative emotion ratio and rising suspicion scores, and the configuration of current features and thresholds. Server fills a text template with numeric values, qualitative descriptions, and contextual information to create a human-readable description of the current analysis state. For example, server may generate a prompt sentence such as:
“To detect early signs of cognitive impairment from user conversations, which sentiment analysis algorithms and feature engineering techniques should be used with the current set of emotion labels, time-series features, and model thresholds to improve sensitivity while limiting false positives?”

The output is a prompt sentence formatted as plain text.

### Step 13

Server transmits the prompt sentence and receives response information from the generative AI model.

Server sends the prompt sentence to a generative AI model via a network interface. The input is the text prompt and any necessary configuration parameters such as maximum response length. Server encodes the prompt, transmits it using a communication protocol, and waits for a response. The generative AI model produces a text response containing suggested feature designs, model structures, or threshold adjustments. Server receives and decodes this response. The output is response information in text form, accessible for further parsing.

### Step 14

Server parses and operationalizes generative AI suggestions.

Server processes the response information to extract concrete configuration values. The input is the text output from the generative AI model. Server applies text parsing rules to identify recommended features (for example, lexical diversity or phrase frequency measures), threshold ranges, or model types (for example, a neural network architecture specification). Server maps these suggestions to internal parameter fields, such as enabling new feature computation modules or adjusting numeric threshold constants. The output is an updated configuration set that encodes modifications to emotion analysis and behavioral pattern detection processing.

### Step 15

Server updates processing modules and models based on new configuration.

Server applies the updated configuration to its runtime environment. The input is the new configuration set derived from the generative AI response. Server activates or deactivates feature extraction functions, alters the set of features the machine learning model uses, or retrains the model with an updated feature space and hyperparameters. Server may also adjust decision thresholds used in behavioral pattern classification. The output is an updated set of processing modules and models that reflect the refinements suggested via the prompt sentence and response cycle.

### Step 16

Server executes notification processing when a threshold is exceeded.

Server evaluates whether a current suspicion score surpasses a configured threshold based on the latest model output. The input is the behavioral pattern detection result and the applicable threshold setting. When the threshold is exceeded, server retrieves user contact data and contact information for designated guardians or healthcare professionals from the storage device. Server composes an alert message summarizing the detected behavioral pattern and contextual statistics, and transmits this message through an electronic communication network to appropriate destinations. The output is an issued notification that reaches one or more recipients via their respective devices.

### Step 17

User and terminal participate in feedback and data enrichment.

User receives the notification or feedback on the terminal and may provide additional information, such as confirming or rejecting the system's suspicion or answering targeted follow-up questions. The input is the alert message and any displayed queries on the terminal. User interacts with the terminal's interface to input responses, and terminal collects this feedback as structured data. Terminal then transmits the feedback to the server, which stores it as labeled outcomes for future model training or threshold calibration. The output is enriched training and evaluation data that can be used by the server to further refine feature extraction, model parameters, and decision rules in subsequent processing cycles.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented systems for monitoring cognitive decline based on user speech typically perform only isolated sentiment detection or keyword spotting on individual utterances. Such systems suffer from several technical limitations. First, they treat each input text segment independently and do not compute long-term temporal features, such as the evolution of emotion tags or changes in linguistic patterns over weeks or months. This results in low detection accuracy and high noise, because transient emotional states are not distinguished from persistent, dementia-related behavioral patterns. Second, conventional architectures do not tightly integrate automatic speech recognition, natural language emotion tagging, feature vector generation, and machine-learning-based risk prediction into a unified processing pipeline optimized for continuous operation on streaming conversational data. As a result, system resources such as CPU time, memory bandwidth, and network usage are not efficiently utilized, and real-time or near-real-time risk estimation for multiple users becomes difficult. Third, existing systems that incorporate generative models often use manually crafted prompts that are not systematically derived from machine-interpretable behavioral features. This causes inconsistent output quality, makes it difficult to reproduce or audit generated explanations, and prevents the generative model from leveraging the full structure of the underlying prediction model's feature space. Fourth, many systems provide only raw scores or unstructured alerts, without generating structured, human-readable reports that clearly summarize which long-term emotional and linguistic trends contributed to the risk assessment, thereby limiting the usefulness of such systems for medical workers and caregivers.

Accordingly, there is a need for a computer-implemented system and method that (i) automatically acquires user voice via a mobile information processing terminal, (ii) converts the voice into character information and attaches emotion tags, (iii) accumulates and transforms these tagged data into behavioral-pattern determination feature vectors capturing temporal and statistical characteristics, (iv) inputs the feature vectors into a trained prediction model to compute a risk index indicative of cognitive function decline, and (v) automatically generates structured prompt sentences and report texts through a generative model in a way that is tightly coupled to the computed feature vectors and risk index. Such a system should improve the technical operation of the computer by enabling more accurate and explainable automated risk estimation, efficient handling of large-scale time-series conversational data, and reproducible generation of natural-language explanations that are aligned with underlying numerical features and model outputs.

The specific processing by the specific processing unit 290 of the data processing device 12 in Application Example 1 is realized by the following means.

The present invention provides a server comprising a processor and a memory storing instructions, wherein the processor is configured to execute the instructions to acquire voice information of a user from a mobile information processing terminal, convert the voice information into character information by using a speech recognition processing resource, perform emotion analysis on the character information by using a natural language processing resource to attach emotion tags indicating emotion type and emotion intensity, store in a data storage device the emotion-tagged character information together with associated time information, calculate, based on a plurality of stored items of emotion-tagged character information, at least one of an occurrence frequency of emotion tags, a temporal change of emotion tags, and a linguistic feature of expressions to generate a behavioral-pattern determination feature vector, input the behavioral-pattern determination feature vector into a prediction model configured by a machine learning processing resource to calculate a risk index representing a behavioral pattern related to cognitive function decline, automatically generate, on the basis of the risk index and the emotion-tagged character information, a prompt sentence to be used as an input to a generative information processing model, cause the generative information processing model to generate explanatory text or a report text summarizing emotional transitions and linguistic tendencies indicative of cognitive function decline, and, when the risk index exceeds a predetermined threshold, transmit notification information including at least the explanatory text or the report text to a related-person terminal or a user terminal. This enables an integrated and technically improved computer-implemented pipeline that efficiently transforms raw voice signals into structured long-term behavioral features, produces more accurate and stable dementia-risk indices using machine learning, and automatically generates consistent, explainable natural-language analyses tightly coupled to the underlying feature vectors, thereby enhancing the performance, scalability, and interpretability of computer systems for monitoring cognitive decline.

The term “voice information” refers to audio data representing sounds produced by a user, including spoken words and utterances, which are captured by an input device such as a microphone and processed by an information processing apparatus.

The term “mobile information processing terminal” refers to a portable electronic apparatus having a communication function and a computation function, such as a smartphone or tablet terminal, which is configured to acquire, locally store, and transmit voice information or character information of a user.

The term “temporary storage region” refers to a memory region or storage area, such as a volatile memory or a non-volatile buffer, in which data such as voice information is stored for a limited period of time before being further processed or transferred to another device or storage.

The term “communication network” refers to a wired or wireless data transmission infrastructure, including at least one of a local area network, a wide area network, and a public packet communication network, through which data is exchanged between the mobile information processing terminal and a server.

The term “speech recognition processing resource” refers to a hardware resource or software resource, or a combination thereof, configured to convert voice information into character information by applying an acoustic model, a language model, or another statistical or rule-based model.

The term “character information” refers to digital data representing linguistic content in a symbolic form, such as text encoded using a character code, obtained by converting voice information or other input information.

The term “natural language processing resource” refers to a hardware resource or software resource, or a combination thereof, configured to analyze character information expressed in a natural language, and to output semantic, syntactic, or emotional information derived from the character information.

The term “emotion analysis” refers to processing that estimates, from character information, at least one of an emotion type and an emotion intensity, based on linguistic features, statistical models, or machine-learning models.

The term “emotion tag” refers to a label or a set of labels associated with character information, the label indicating an inferred emotional state, such as anxiety, sadness, or confusion, and optionally indicating a degree or intensity of the emotional state.

The term “data storage device” refers to a non-transitory computer-readable storage medium or a storage subsystem, such as a magnetic disk device, a semiconductor memory device, or a distributed storage system, configured to store data including character information, emotion tags, and associated time information.

The term “time information” refers to data indicating a time at which an event occurred or data was acquired, such as a timestamp or a time interval, which is associated with corresponding voice information or character information.

The term “behavioral-pattern determination feature vector” refers to a numerical vector or structured data representing at least one of an occurrence frequency of emotion tags, a temporal change of emotion tags, and a linguistic feature of expressions, derived from a plurality of items of emotion-tagged character information, and used as an input to a prediction model.

The term “linguistic feature” refers to a property of character information derived from analysis of language use, including at least one of word frequency, sentence length, lexical diversity, syntactic structure, or occurrence of specific phrases, which is usable as an element of a feature vector.

The term “prediction model” refers to a computational model constructed by machine learning or statistical learning, which receives a behavioral-pattern determination feature vector as input and outputs at least one of a classification result, a probability value, or a risk index regarding a behavioral pattern.

The term “machine learning processing resource” refers to a hardware resource, a software resource, or a combination thereof, configured to train and execute a prediction model using training data, feature vectors, and numerical computation, including at least one of a processor, a graphics processing unit, and a machine-learning library.

The term “risk index” refers to a numerical value or score output by the prediction model, indicating a degree of likelihood or severity of a behavioral pattern related to cognitive function decline, such as a probability or normalized risk level.

The term “cognitive function decline” refers to a decrease in one or more cognitive abilities of a user, including at least one of memory, attention, language, and executive function, which may be associated with a neurological disorder such as dementia.

The term “generative information processing model” refers to a computational model, such as a generative artificial intelligence model or a large language model, which receives an input including a prompt sentence and optionally structured data, and generates natural-language text or other data that did not explicitly exist in the input.

The term “prompt sentence” refers to text data formulated to instruct a generative information processing model to perform a specific task, the text including at least one of a description of required output, contextual information, and parameters related to analysis or reporting.

The term “explanatory text” refers to natural-language text generated to describe or clarify an analysis result, including an interpretation of a risk index, an outline of contributing factors, or a qualitative explanation of detected behavioral patterns.

The term “report text” refers to a structured natural-language document generated for presentation to a human reader, summarizing at least one of transitions of emotion tags, frequencies of specific utterances, and tendencies of linguistic expressions, together with conclusions or recommendations.

The term “notification information” refers to data transmitted to a related-person terminal or a user terminal, including at least one of an alert, an explanatory text, a report text, and a risk index, which indicates a result of analysis related to cognitive function decline.

The term “related-person terminal” refers to an information processing apparatus operated by a person related to the user, such as a medical worker or a caregiver, which is configured to receive and display notification information or report text.

The term “user terminal” refers to an information processing apparatus operated by the user whose voice information is acquired, which is configured to receive and display notification information, explanatory text, or report text.

The term “feedback information” refers to data provided from the user or a related person, indicating at least one of correctness, relevance, or usefulness of analysis results or report texts, and used to update or retrain the prediction model or to adjust processing parameters.

In one embodiment, a system includes at least one server, at least one mobile information processing terminal (hereinafter “terminal”), and at least one data storage device interconnected via a communication network. The server includes a processor and a memory storing instructions, and the terminal includes a processor, a microphone, a display, and a wireless communication interface.

The user carries the terminal in daily life and speaks naturally in front of the terminal. The terminal uses an audio input component of an operating system, such as an audio capture API of a mobile operating system, to acquire voice information of the user. The terminal samples the analog speech signal from the microphone at a predetermined sampling rate, for example 16 kHz, and quantizes the signal into digital samples. The terminal encodes the samples using a compression codec such as Advanced Audio Coding or another low-bitrate codec, and writes the compressed audio stream into a temporary storage region in a non-transitory memory, such as internal flash memory of the terminal.

The terminal associates metadata with each audio segment, including at least a user identifier, a device identifier, a start timestamp, and an audio duration. The terminal stores the metadata together with a file path or object key of the audio segment as a structured record, for example in a key-value store or a lightweight embedded database within the terminal. The terminal transmits the audio segment and its metadata to the server via a communication network using a secure protocol such as HTTPS, by means of a network stack provided by the operating system.

The server receives the audio segment in a web service module implemented, for example, using a network framework on a server operating system. The server stores the received audio segment in a data storage device, such as a network-attached storage or cloud object storage. The server generates a unique audio identifier and registers a mapping between the audio identifier, the associated metadata, and the storage location in a relational database or a document-oriented database.

The server converts the voice information into character information by using a speech recognition processing resource. In one embodiment, the server uses a speech-to-text engine running on the server or provided by a cloud-based recognition service. The server passes the audio identifier and configuration parameters, such as language code, acoustic model type, and punctuation setting, to a speech recognition module. The speech recognition module loads an acoustic model and a language model, for example a deep neural network acoustic model and an n-gram or neural language model, and performs decoding by computing acoustic likelihoods and language probabilities over a recognition lattice. The server obtains recognized text segments with associated confidence scores and arranges them into a single piece of character information for each audio segment. The server stores the character information and the confidence scores in the database, linked to the original audio identifier.

The server performs emotion analysis on the character information by using a natural language processing resource. In one embodiment, the server uses a sentiment and emotion analysis engine that implements feature extraction for natural language, including tokenization, part-of-speech tagging, dependency parsing, and embedding generation. The server applies a trained classifier, such as a neural network classifier built on top of contextual word embeddings, to estimate probabilities for a plurality of emotion types (for example, anxiety, sadness, joy, anger, confusion) and corresponding intensities. The server converts these probabilities into emotion tags by applying predefined rules, such as selecting the top-k emotions exceeding a threshold, and quantizing intensities into levels (for example, low, medium, high). The server associates each piece of character information with one or more emotion tags and stores the resulting emotion-tagged character information, together with associated time information, in the data storage device.

The server constructs a behavioral-pattern determination feature vector for each user by aggregating a plurality of items of emotion-tagged character information over a defined time window, such as a week or a month. The server retrieves all records for a given user and time window from the data storage device and computes statistical features, including occurrence frequencies of each emotion tag, co-occurrence counts of specific tag combinations (for example, anxiety and forgetfulness), moving averages and trend slopes of emotion intensities over time, and distributional measures such as variance and skewness. The server also computes linguistic features from the character information, such as average sentence length, type-token ratio, frequency of question sentences, frequency of temporal expressions, and occurrence of phrases associated with memory issues.

The server concatenates the statistical emotion features and linguistic features into a numerical vector of fixed dimension, which serves as the behavioral-pattern determination feature vector. In one embodiment, the server normalizes each feature dimension by subtracting a mean and dividing by a standard deviation obtained from training data. The server optionally applies dimensionality reduction techniques, such as principal component analysis, to reduce correlation and improve computational efficiency.

The server inputs the behavioral-pattern determination feature vector into a prediction model configured by a machine learning processing resource. In one embodiment, the prediction model is a neural network implemented by a machine learning framework such as a tensor computation framework. The neural network may have a feed-forward architecture with multiple fully connected layers, for example an input layer, two or more hidden layers with rectified linear unit activation functions, and an output layer with a sigmoid activation function. The server loads trained weights and biases of the neural network from a model file stored in the data storage device into the memory at startup.

The server computes the risk index representing a behavioral pattern related to cognitive function decline by multiplying the feature vector by the network weights, applying the activation functions layer by layer, and obtaining an output value between zero and one as the risk index. In another embodiment, the prediction model may be a recurrent neural network or a transformer-based model that explicitly models temporal sequences of feature vectors. The server trains the prediction model offline on historical data labeled by experts, using a loss function such as binary cross-entropy, a gradient-based optimization algorithm such as stochastic gradient descent with momentum or adaptive moment estimation, and regularization techniques such as dropout and L2 weight decay. The server periodically retrains or fine-tunes the prediction model using newly accumulated emotion-tagged data and user feedback information, thereby updating the mapping between feature vectors and risk indices and improving detection accuracy.

The server generates a prompt sentence to be used as an input to a generative AI model on the basis of the risk index and the emotion-tagged character information. The server composes the prompt sentence according to a predefined template that includes: an instruction part specifying a role and task for the generative AI model; a data summary part describing key statistics from the behavioral-pattern determination feature vector; and a contextual part including representative utterances and emotion tags. For example, the server may generate a prompt sentence such as:
“You are a medical support assistant. Analyze the following emotion-tagged utterances and explain in plain language whether there are patterns that may indicate early dementia. Data: [list of utterances with timestamps and emotion tags].” or
“Generate a short report for a caregiver based on the following user statements and emotion tags. Highlight repeated forgetfulness, anxiety, or confusion, and suggest whether a medical consultation should be considered.”

The server sends the prompt sentence, along with structured data derived from the feature vector, to a generative AI model executed either on the same server or on a remote computing resource accessible via an application programming interface. The generative AI model may be a large language model based on a transformer architecture, pre-trained on large text corpora and optionally fine-tuned for medical support tasks. The server receives the generated explanatory text or report text and stores it in the data storage device associated with the corresponding risk index and time window.

The server compares the risk index to a predetermined threshold stored in configuration data. When the risk index exceeds the threshold, the server generates notification information including at least the explanatory text or report text and the risk index value. The server transmits the notification information to a related-person terminal, such as a workstation or tablet used by a medical worker or caregiver, and optionally to the user terminal, using a push notification service, an e-mail protocol, or another communication mechanism. The terminal displays the notification information on a graphical user interface, allowing the user or related person to review the analysis and suggested actions.

The server thereby improves computer technology itself in multiple respects. By transforming raw audio streams into structured, time-series behavioral-pattern determination feature vectors optimized for machine learning, the server reduces the volume of data that must be repeatedly processed, lowering communication and storage overhead. The statistical aggregation and normalization steps reduce noise and variance in emotional and linguistic measurements, enabling the prediction model to converge faster and to produce more stable risk indices than systems that operate only on individual utterances. The design of the feature vector and the neural network architecture is specifically tailored to capture long-term patterns of emotion transitions and linguistic change, which are not practically tractable by manual human observation at scale.

The server also improves computational efficiency by separating local processing and central processing. The terminal performs only audio capture and initial encoding, while the server performs heavy numerical computations, such as speech recognition, feature extraction, and neural network inference, on hardware optimized for such tasks, including multi-core processors and graphics processing units. This division reduces power consumption on the terminal and allows the system to handle many users in parallel on the server.

The server uses a non-conventional integration of discriminative and generative models. Instead of manually crafted prompts, the server automatically constructs prompt sentences from machine-interpretable feature vectors and risk indices according to deterministic rules. This produces reproducible and auditable inputs to the generative AI model, which in turn generates human-readable explanations aligned with underlying numerical features. This tight coupling between statistical computation and natural-language generation yields a technical effect of improved interpretability and debuggability of machine-learning outputs, which cannot be obtained by conventional systems that merely display raw scores or isolated classification labels.

The server further employs feedback information from users or related persons. When the user or caregiver views a report and indicates whether the described behavior pattern is accurate, the terminal sends feedback data to the server. The server stores the feedback as additional labels for retraining the prediction model. The server may modify loss weights or sample selection strategies based on feedback, emphasizing misclassified or uncertain cases. This closed feedback loop results in continuous improvement of model accuracy and robustness, which is a technical enhancement over static models that are not updated after deployment.

In alternative embodiments, the server may use different machine-learning architectures, such as gradient-boosted decision trees or probabilistic graphical models, to implement the prediction model. The server may also vary the set of features included in the behavioral-pattern determination feature vector, such as adding acoustic features extracted directly from the audio signal (for example, pitch variation, speech rate, pause duration) to capture paralinguistic cues of cognitive decline. The terminal may implement additional pre-processing, such as on-device noise suppression and voice activity detection, to improve speech recognition accuracy and reduce unnecessary data transmission.

The user operates the system in various usage scenarios. For example, the user may install an application on the terminal that automatically records certain categories of conversation, such as phone calls, diary-like voice memos, or conversations with a digital assistant. The user may grant consent and configure recording schedules and privacy settings. The user may review summarized trends in emotion tags and risk indices displayed on the terminal, and may choose to share these summaries with family members or medical providers via the system.

Through these embodiments, the server, the terminal, and the user cooperate to implement a concrete technical solution: the system acquires real-world voice signals, transforms them into structured, temporally aggregated feature representations, executes specialized machine-learning models and generative AI models, and controls notification and display on physical devices. This solution goes beyond mere automation of human judgment and instead provides an improved computer-implemented mechanism for efficient data handling, higher detection accuracy, stable risk estimation, and explainable reporting in the context of monitoring cognitive function decline.

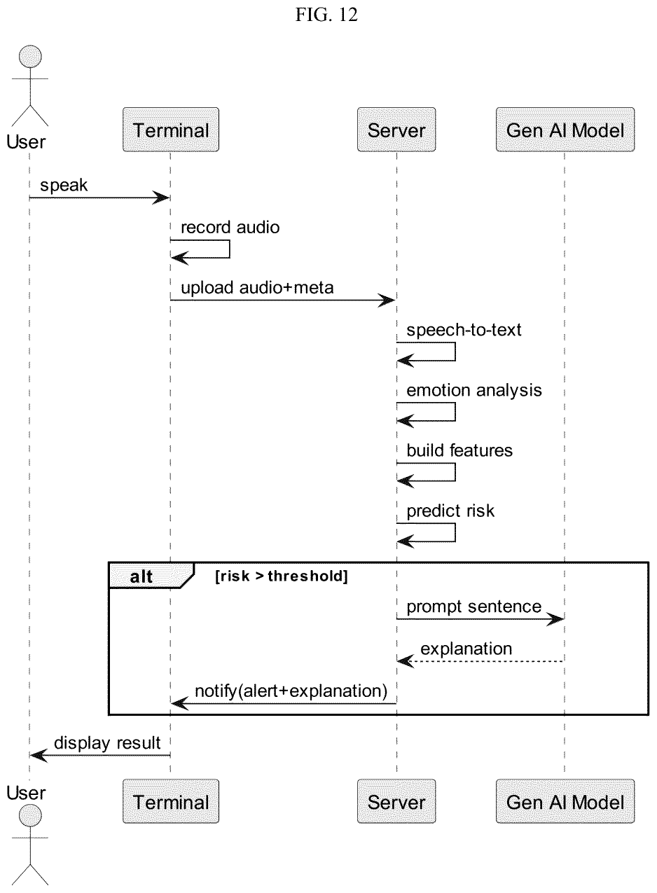

The following describes the processing flow using FIG. 12.

### Step 1

The user speaks naturally in front of the terminal during daily life.

The terminal uses a microphone and an operating system audio API to capture the user's analog voice signal as an input.

The terminal samples the analog signal at a predetermined sampling rate (for example, 16 kHz) and quantizes it into digital audio frames.

The terminal encodes the digital audio frames with an audio codec (for example, a low-bitrate compression codec) and writes the encoded audio stream into a temporary file in internal storage.

The terminal generates metadata including a user identifier, a device identifier, a start timestamp, and a duration, and associates the metadata with the audio file path.

The output of Step 1 is a compressed audio file and corresponding metadata stored in a temporary storage region on the terminal.

### Step 2

The terminal checks network connectivity as an input condition by querying a network status API of the operating system.

The terminal, when connectivity is available, prepares an upload request that includes the audio file and metadata generated in Step 1 as input data.

The terminal opens an HTTPS connection to the server's endpoint and transmits the audio file and metadata using a POST request with appropriate authentication information.

The server receives the HTTP request, validates authentication tokens, verifies the audio format and file size, and rejects malformed or unauthorized requests.

The server stores the received audio file in a data storage device and registers a record in a database that maps a newly assigned audio identifier to the file location and associated metadata.

The output of Step 2 is an audio identifier and a stored audio object managed by the server.

### Step 3

The server takes as input the audio identifier and associated stored audio file produced in Step 2.

The server invokes a speech recognition processing resource, specifying recognition parameters such as language code and punctuation options.

The server loads or accesses an acoustic model and a language model, and feeds audio frames from the stored audio file into the recognition engine.

The server performs data operations including feature extraction (for example, computing spectral features), probability computation using the acoustic model, and decoding using the language model to obtain the most likely text sequence.

The server outputs character information (recognized text) and confidence scores for the transcription, and stores these in the database linked to the audio identifier.

The output of Step 3 is character information representing the user's utterance and associated recognition confidence values.

### Step 4

The server uses the character information from Step 3 as input to a natural language processing resource for emotion analysis.

The server tokenizes the text, performs part-of-speech tagging, and generates word or sentence embeddings using a language representation model.

The server inputs these embeddings into an emotion classifier model that computes probabilities for multiple emotion categories and their intensities based on learned parameters.

The server applies thresholding and mapping rules to convert the probability vector into discrete emotion tags and quantized intensity levels.

The server associates the emotion tags and intensity levels with the original character information and stores the resulting emotion-tagged character information and time information in the data storage device.

The output of Step 4 is emotion-tagged character information for each utterance, including emotion types and intensities.

### Step 5

The server retrieves, as input, a plurality of items of emotion-tagged character information for a specific user over a defined time window from the data storage device.

The server groups the records by user and time interval and computes statistical measures such as the count and relative frequency of each emotion tag, co-occurrence frequencies of tag combinations, and time-series summaries such as moving averages and trend slopes.

The server extracts linguistic features from the character information, such as average sentence length, vocabulary diversity, and frequency of forgetfulness-related expressions.

The server normalizes each feature dimension using pre-stored mean and variance values and concatenates the normalized statistical and linguistic features into a fixed-length numerical vector.

The server outputs this numerical vector as a behavioral-pattern determination feature vector representing the user's behavioral pattern within the time window.

The output of Step 5 is a behavioral-pattern determination feature vector for each user and time window.

### Step 6

The server uses the behavioral-pattern determination feature vector from Step 5 as input to a prediction model implemented by a machine learning processing resource.

The server loads model parameters, such as neural network weights and biases, from a stored model file into memory.

The server feeds the feature vector through the model, performing matrix multiplications, activation function evaluations, and layer-by-layer forward propagation.

The server computes a scalar output value between zero and one that represents a risk index for a behavioral pattern related to cognitive function decline.

The server writes the risk index and the corresponding feature vector identifier into the database for later reference and analysis.

The output of Step 6 is a risk index associated with the user and the analyzed time window.

### Step 7

The server compares, as an input condition, the risk index from Step 6 to a predetermined threshold value stored in configuration data.

The server, when the risk index exceeds the threshold, collects supporting data including representative emotion-tagged character information, summary statistics from the feature vector, and recent trend indicators.

The server constructs a prompt sentence by inserting the collected data into a predefined template that instructs a generative AI model to analyze and explain the risk.

For example, the server may generate a prompt sentence such as: “You are a medical support assistant. Analyze the following emotion-tagged utterances and explain in plain language whether there are patterns that may indicate early dementia. Data: [list of utterances with timestamps and emotion tags].”

The server sends the prompt sentence and, optionally, structured feature data to the generative AI model via an application programming interface and receives as output an explanatory text or a report text summarizing emotional transitions and linguistic tendencies.

The output of Step 7 is an explanatory text or report text generated by the generative AI model based on the prompt sentence and the behavioral features.

### Step 8

The server uses the risk index from Step 6 and the generated explanatory or report text from Step 7 as input to a notification generation module.

The server formats notification information that includes at least the risk index, a summary of key contributing features, and the explanatory or report text, and associates the notification with identifiers of a user terminal and a related-person terminal.

The server selects an appropriate communication channel, such as a push notification service or an e-mail protocol, and transmits the notification information to the designated terminals.

The terminal receives the notification as input from the communication service and displays the content on a user interface, including textual explanation and suggested actions.

The user views the displayed notification and may optionally provide feedback, such as confirming or correcting the described behavioral pattern, which the terminal sends back to the server for use as additional training or calibration data.

The output of Step 8 is the presentation of risk information and explanatory text on the terminals and, optionally, feedback data returned from the user or related person to the server.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional health-and behavior-monitoring systems typically process bio-signal data and behavior logs in isolation from user-generated voice and language data. In many implementations, sensor streams from wearable devices are merely thresholded or statistically filtered, and alerts are triggered when simple numerical limits are exceeded. Such architectures suffer from several technical limitations.

First, the underlying data processing pipelines are not optimized to handle heterogeneous, time-series physical activity information and unstructured voice information in an integrated manner. Raw sensor streams often contain missing values, inconsistent timestamps, and outliers. If these issues are not systematically resolved within the data path, downstream models execute on noisy or misaligned data, which degrades anomaly detection accuracy and leads to unstable system behavior. This, in turn, produces a large number of false positives and false negatives, and requires additional computing cycles for repetitive re-processing and manual tuning.

Second, emotion information derived from user speech is rarely fused with physical activity information at the system level. Existing systems that perform speech-based emotion analysis typically operate in a siloed manner, without feeding emotion identification information back into the health-or cognition-monitoring pipeline. As a result, the processor cannot exploit correlations between emotional states and physical activity patterns to refine detection logic. This separation limits the ability of the system to distinguish between benign deviations and meaningful abnormal behavior, thereby constraining the effective use of computational resources and model capacity.

Third, many deployments rely on static, hand-crafted thresholds and fixed decision rules that do not adapt over time. As a user's behavior pattern and baseline physical condition gradually change, such static configurations become outdated. The processor continues to apply the same criteria to increasingly unrepresentative data distributions, which deteriorates the utility of anomaly scores and increases the computational cost of compensating processes, such as repeated manual recalibration or frequent re-training. This lack of dynamic adaptation represents an inefficiency in the use of memory and processing resources within the computing environment.

Fourth, anomaly detection and alerting logic in conventional systems are often decoupled from higher-level generative models capable of proposing new detection strategies or personalized notification content. Without a structured mechanism to generate and apply prompt sentences to a generative information processing model, the processor cannot systematically exploit generative model outputs to reconfigure thresholds, message templates, and analysis parameters. This leads to a rigid architecture in which software components must be manually reprogrammed to incorporate improvements, thereby increasing maintenance overhead and limiting scalability.

Accordingly, there is a need for technological improvements in computer systems that (i) perform structured preprocessing and storage of physical activity information, (ii) integrate emotion identification information derived from speech with anomaly detection results from a machine learning model, (iii) dynamically update decision criteria based on long-term behavior changes, and (iv) leverage a generative information processing model via prompt sentences to automatically refine analysis request information and notification content. By addressing these issues at the system architecture and data-processing levels, the invention aims to improve the accuracy, adaptability, and computational efficiency of cognitive-state monitoring performed by a processor-based system.

The specific processing by the specific processing unit 290 of the data processing device 12 in Example 2 is realized by the following means.

The present invention provides a server comprising a processor configured to acquire, via a mobile terminal, voice information and physical activity information of a user; to convert the voice information into character information by performing a speech recognition process and to apply natural language processing to the character information to extract emotion information and assign emotion identification information; to perform numerical processing and preprocessing on the physical activity information, the preprocessing including at least unifying time information, complementing missing values, correcting outliers, and normalizing numerical values; to store the preprocessed physical activity information as structured information in a storage device; to use a machine learning model on the structured information to learn a normal behavior pattern of the user and calculate abnormal state information according to a degree of deviation from the normal behavior pattern; to generate a prompt sentence including analysis request information for analyzing a sign of cognitive function decline on the basis of the emotion identification information and the abnormal state information and to instruct a generative information processing model to receive the prompt sentence as input; and to, when the abnormal state information satisfies a predetermined determination criterion, acquire contact information of a notification target and transmit warning information to the contact information. This enables integrated, machine-executable processing of heterogeneous time-series and language data, adaptive anomaly detection with dynamically maintainable thresholds, and automatic refinement of analysis and notification behavior via generative model feedback, thereby improving the technical performance, accuracy, and configurability of computer-implemented cognitive-state monitoring.

The term “processor” refers to a hardware or virtual computing element, such as a central processing unit or an execution core, that executes instructions to perform data acquisition, preprocessing, model inference, prompt generation, and notification control in the system.

The term “mobile terminal” refers to a portable electronic apparatus, such as a handheld communication device, configured to communicate with a wearable or other sensor device and to transmit acquired data to the server over a communication network.

The term “information processing apparatus” refers to an electronic computing environment, including hardware resources and software components, that is configured to execute programs for handling voice information, physical activity information, and related analysis operations.

The term “voice information” refers to audio data representing spoken utterances of a user, captured by a microphone of the mobile terminal or another recording device, and suitable for input to a speech recognition process.

The term “physical activity information” refers to measurement data indicative of a user's bodily state or movement, including at least heart rate, step count, activity level, or similar biosignal and motion-related parameters obtained from one or more sensors.

The term “user” refers to an individual whose voice information and physical activity information are acquired and analyzed by the system.

The term “speech recognition process” refers to a computational procedure that converts voice information into character information by detecting linguistic units such as phonemes, words, and phrases using an acoustic and language model.

The term “character information” refers to textual data, represented in a machine-readable character encoding, that is obtained as a result of applying the speech recognition process to voice information.

The term “natural language processing” refers to a set of computational techniques for analyzing character information, including tokenization, parsing, sentiment estimation, and semantic interpretation, to derive structured features from unstructured text.

The term “emotion information” refers to data that represents an inferred emotional state of the user, such as positive, negative, neutral, or more granular affective categories, derived by applying natural language processing to character information.

The term “emotion identification information” refers to a structured representation, such as a label, tag, or score, assigned to the character information or to a time segment thereof, indicating a classified emotional state of the user.

The term “numerical processing” refers to computational operations performed on numerical values in the physical activity information, including at least arithmetic operations, filtering, scaling, and statistical transformations.

The term “preprocessing” refers to a sequence of data preparation operations applied to raw physical activity information, including unifying time information, complementing missing values, correcting outliers, and normalizing numerical values, to produce data suitable for machine learning.

The term “time information” refers to metadata associated with physical activity information or voice information that indicates a time or date of acquisition or validity, including timestamps or time indices.

The term “complementing missing values” refers to a procedure of estimating and filling in absent measurement values in a time series by interpolation, forward filling, backward filling, or other imputation methods.

The term “correcting outliers” refers to a procedure for detecting and adjusting data points that deviate beyond a predetermined range or statistical criterion, in order to reduce the impact of measurement errors or artifacts.

The term “normalizing numerical values” refers to scaling or transforming numerical data into a standardized range or distribution, such as by min-max scaling or z-score standardization, to improve the stability of subsequent computational processing.

The term “structured information” refers to data organized into a defined schema, such as records with named fields, tables, or arrays, enabling indexed storage, retrieval, and processing in a storage device or database.

The term “storage device” refers to a physical or virtual memory component, such as a database system or non-volatile memory, configured to store structured information and related metadata for later access by the processor.

The term “machine learning model” refers to a computational model whose parameters are learned from training data and that is configured to receive structured information as input and output a prediction, classification, or score, including but not limited to neural networks, regression models, and anomaly detection models.

The term “normal behavior pattern” refers to a statistical or model-based representation of typical physical activity information of a user over a reference period, as learned by the machine learning model from historical data.

The term “abnormal state information” refers to data indicating a degree and type of deviation of current physical activity information from the normal behavior pattern, including anomaly scores, flags, or categorical labels.

The term “prompt sentence” refers to a machine-readable text sequence that encodes analysis request information, context, or instructions, intended as input to a generative information processing model.

The term “analysis request information” refers to content within the prompt sentence that specifies what type of analysis, inference, or recommendation the generative information processing model is requested to perform.

The term “generative information processing model” refers to a computational model configured to generate text or other data in response to a prompt sentence, based on learned patterns from training data, and implemented using machine learning techniques such as generative language modeling.

The term “sign of cognitive function decline” refers to an inferred condition indicating a possible reduction in cognitive abilities, estimated by analyzing combinations of emotion identification information and abnormal state information over time.

The term “predetermined determination criterion” refers to one or more configurable conditions or thresholds used by the processor to judge whether abnormal state information indicates an event that should trigger further analysis or notification.

The term “notification target” refers to an entity, such as a person or organization associated with the user, that is designated to receive warning information when the predetermined determination criterion is satisfied.

The term “contact information” refers to addressing data used for communication with a notification target, including at least a telephone number, electronic mail address, or messaging account identifier.

The term “warning information” refers to content of a notification generated by the processor, indicating that an abnormal condition or a sign of cognitive function decline has been detected and optionally including recommended actions.

The term “long-term change in a behavior pattern” refers to a variation in the user's behavior characteristics measured over an extended period, as determined by comparing current abnormal state information with past structured information.

The term “past structured information” refers to structured information previously stored in the storage device, representing historical physical activity information and associated analysis results for the user.

The term “determination criterion” refers to a rule set or numerical threshold configuration used by the processor when evaluating abnormal state information and deciding whether to raise an alert or initiate further processing.

The term “proposal information” refers to data output from the generative information processing model, including suggestions for modifying analysis request information, thresholds, or notification messages.

The term “setting information” refers to configuration data generated by the processor based on proposal information, used to change parameters such as thresholds of the machine learning model or content templates of warning information.

The term “threshold of the machine learning model” refers to a numerical value or set of values used to interpret an output of the machine learning model as normal or abnormal, and to control sensitivity and specificity of anomaly detection.

The term “notification content” refers to the substantive text or message body included in warning information that is transmitted to a notification target.

In one embodiment, a server cooperates with a terminal and a user to implement the claimed system. The server includes at least one processor, a memory, and a network interface. The terminal includes a processor, a memory, a microphone, a communication interface, and optionally a display. The user carries or wears a sensing device, such as a wearable sensor, that measures physical activity information and transmits the information to the terminal.

The user wears a wearable sensor that acquires physical activity information, including heart rate, step count, and activity level, by means of integrated biosensors and motion sensors. The user provides voice information by speaking near a microphone of the terminal or another audio input device. The terminal captures the voice information as digital audio data and obtains the physical activity information via short-range wireless communication, such as Bluetooth Low Energy or wireless local area communication.

The terminal transmits both the voice information and the physical activity information to the server over a network. The server receives the information and stores it temporarily in a memory, such as a random access memory, for further processing. The server uses a speech recognition engine, implemented for example with a language-independent automatic speech recognition library, to convert the voice information into character information. The server applies natural language processing software, such as a text processing framework that performs tokenization, part-of-speech tagging, and sentiment analysis, to the character information in order to extract emotion information and assign emotion identification information. The emotion identification information is represented as discrete tags and continuous scores, and the server stores these in a structured form, such as records in a database table.

The server processes the physical activity information using a numeric computing library, such as a numerical array library or a data frame library, to perform preprocessing. The server unifies time information by converting all timestamps to a common reference time zone and a standardized format and by aligning values onto a fixed time grid. The server complements missing values using interpolation methods, such as linear interpolation or previous-value carry-forward, depending on the type of signal. The server corrects outliers by detecting values that deviate from a statistical distribution, such as values exceeding a multiple of the standard deviation or falling outside a percentile range, and then replacing them with estimated values based on surrounding measurements. The server normalizes numerical values by applying scaling, such as min-max scaling or z-score normalization, so that the physical activity information is mapped into ranges appropriate for input into a machine learning model.

The server stores the preprocessed physical activity information as structured information in a storage device, such as a relational database system. The structured information uses a schema with fields that include user identifiers, timestamps, heart rate values, step counts, activity levels, data quality flags, and derived features. The structured information is indexed by user and by time to enable efficient retrieval of time-series segments relevant for model processing. The server thus maintains a time-series database optimized for sequential access and batch queries, which improves retrieval speed and reduces the number of disk accesses required to assemble input sequences for the machine learning model.

The server uses a machine learning model to learn a normal behavior pattern of the user from the structured information. In one embodiment, the server implements a neural network architecture specialized for time-series anomaly detection, such as a recurrent neural network, a long short-term memory network, or a gated recurrent unit network. The server constructs input sequences of a fixed length, such as sequences of several hours of heart rate and activity features, and feeds them into an encoder-decoder model or autoencoder configured to reconstruct normal patterns. The server trains the model offline or in an online-learning mode by minimizing an error function, such as mean squared error between input sequences and reconstructed sequences. The server updates model weights using gradient-based optimization, such as stochastic gradient descent or a variant such as Adam. The server may apply data augmentation techniques, for example by adding small noise or by time-shifting sequences, to increase robustness against minor fluctuations.

The server calculates abnormal state information by applying the trained machine learning model to newly acquired structured information. The server computes reconstruction errors or anomaly scores for each time step or sequence window. The server compares the anomaly scores with thresholds that may be set per-feature and per-user. The server may use percentile-based thresholds, such as a threshold equal to a high percentile of reconstruction error observed during training on normal data. The server may also apply a learned classifier layer on top of encoded features to output a categorical label of normal or abnormal. The abnormal state information is represented as a combination of numeric anomaly scores, binary flags, and categorical labels, and is stored back into the structured information.

The server integrates the emotion identification information with the abnormal state information. The server correlates time-aligned emotion identification information with the corresponding time segments of physical activity information. The server aggregates these correlated data into a feature set that describes both emotional states and physical states of the user over a period. The server generates a prompt sentence that includes analysis request information for analyzing signs of cognitive function decline. The prompt sentence encodes, in natural language, a description of recent abnormal state information, emotion identification information, and relevant historical statistics. An example of such a prompt sentence is:
“The server has detected a sustained decrease in the user's activity level over the past three days, accompanied by frequent negative emotion tags extracted from the user's speech. Please analyze whether these combined patterns indicate an early sign of cognitive function decline, and propose additional features or threshold adjustments to improve the detection accuracy.”

The server transmits the prompt sentence to a generative AI model executed in a separate processing environment or service. The server receives proposal information, such as recommendations for new feature combinations, modified thresholds, or refined categories of abnormal states. The server generates setting information based on the proposal information and updates internal configuration values, such as the threshold of the machine learning model, the window lengths for time aggregation, or the template of warning information messages. In this way, the server uses the generative AI model not merely as a text generator, but as a component in a closed-loop configuration update system that continuously refines internal parameters without manual coding.

The server determines whether to send warning information to a notification target by comparing abnormal state information with a predetermined determination criterion. The server may implement the determination criterion as a rule set that considers both anomaly scores and emotion identification information, such as rules that require both a sustained high anomaly score and a certain density of negative emotion tags. The server evaluates long-term changes in behavior patterns by comparing recent abnormal state information with past structured information. The server computes trend measures, such as moving averages or slopes of feature trajectories, and adjusts thresholds dynamically if it detects gradual shifts in the baseline behavior. This adaptive updating reduces false positives caused by stable lifestyle changes and improves the stability and efficiency of the anomaly detection algorithm over time.

The server retrieves contact information of a notification target, such as a family member or a caregiver, from the storage device and composes warning information. The server uses templates that are adapted based on the current type of abnormal state and the configuration provided by the generative AI model. An example of a warning message is:
“The user's recent activity level has decreased significantly compared to the normal behavior pattern, and speech analysis indicates increased negative emotional state. Please consider contacting a medical professional to evaluate the user's cognitive condition.”

The server transmits the warning information via a messaging interface, such as a telecommunication API or an electronic mail server. The terminal receives the warning information and may display it on a screen, provide audio output, or offer interactive actions, such as buttons for calling a professional. The user or the notification target can thus respond to objectively detected abnormalities rather than relying solely on subjective observation.

The server achieves technical improvements over conventional systems through several mechanisms. Because the server performs structured preprocessing, including time unification and outlier correction, before feeding data into the machine learning model, the server reduces noise at the input and allows the model to operate with smaller, more stable weight values. This leads to faster convergence in training, reduced computational load for inference, and increased robustness to missing or corrupted data. The server's use of a time-series oriented database schema and indexed access patterns reduces disk input/output operations and shortens the latency between data acquisition and anomaly detection. The server's integration of emotion identification information and physical activity information allows the model to operate in a higher-dimensional feature space that is not easily handled by human operators through manual rules, thereby increasing detection accuracy without a linear increase in human tuning effort.

The server uses non-conventional processing steps by generating intermediate representations, such as emotion identification information and structured abnormal state information, and by feeding them into both discriminative models and a generative AI model. The server uses the outputs of the generative AI model not as final decisions but as configuration suggestions that adjust machine parameters, such as thresholds and feature weights. This loop results in a form of meta-optimization of the system architecture itself, in which the generative AI model proposes changes that the server encodes as deterministic configuration updates. Such a configuration feedback mechanism is technically different from simply automating human decision-making because it directly alters internal machine state to improve computational performance metrics, such as precision, recall, and processing time.

The server thus improves computational efficiency by avoiding excessive false alarms. When thresholds are tuned automatically based on long-term behavior and prompt-based suggestions, the server reduces the number of unnecessary alert computations and message transmissions. The server can compress stored features and discard redundant raw data after sufficient training, reducing storage usage. The server may also compress time series using dimensionality reduction techniques, such as principal component analysis applied to encoded features, to reduce model input size. As a result, the server performs required analysis with fewer arithmetic operations and smaller memory footprints, which is a technical improvement in the operation of the computer system itself.

The server can implement alternative embodiments of the machine learning model. In one embodiment, the server uses a convolutional neural network applied to time-series segments encoded as multi-channel sequences of heart rate, motion features, and derived statistics. In another embodiment, the server uses a hybrid model that first applies a feature extractor, such as a temporal convolution layer, and then uses a recurrent layer to capture longer-term temporal dependencies. The server chooses hyperparameters such as number of layers, number of units per layer, learning rate, and batch size based on system constraints and available computational resources. The server may also maintain multiple models per user or per user group and select a model with the best validation performance for deployment.

The server may implement alternative embodiments of the generative AI model interaction. In one variation, the server constructs prompt sentences that explicitly encode structured metadata, such as “For user X, anomaly score series: [. . . ], emotion tags over last 7 days: [. . . ]. Propose three new threshold ratios and justify them.” Another example of a prompt sentence is:
“The server stores preprocessed physical activity information in a structured database and uses a neural network-based anomaly detection model to monitor user behavior. Based on the following statistics and error distributions, please suggest improved rules for combining anomaly scores and emotion tags to reduce false positives while maintaining high sensitivity to early cognitive decline.”

The server can thus exploit the generative AI model as an external optimization and explanation engine that proposes new rule sets. The server then encodes the accepted rules into deterministically evaluated conditions that run within the main processing pipeline. This approach creates a clear technical separation between suggestion generation and rule execution, while still enabling the system to evolve without manual rewriting of code.

The terminal can also implement variations in how it interacts with the server. In one embodiment, the terminal performs preliminary preprocessing, such as down-sampling or compressing activity data before transmission, in order to reduce communication load. In another embodiment, the terminal caches data during network outages and synchronizes with the server when connectivity is restored. These variations reduce network bandwidth consumption and improve robustness of the overall system, which are technical effects at the communication and system-integration level.

The user may interact with the system through a graphical interface provided by the terminal. The user can review their own activity trends and emotional trend summaries, but the critical technical processing takes place on the server side in the form of structured preprocessing, model training and inference, threshold adaptation, and prompt-based configuration updates. The described embodiments demonstrate how the server, the terminal, and the user cooperate to implement a computer-implemented technique that improves data quality, model performance, computation efficiency, and alert accuracy, thereby providing a concrete improvement in the operation of computer systems used for monitoring signs of cognitive function decline.

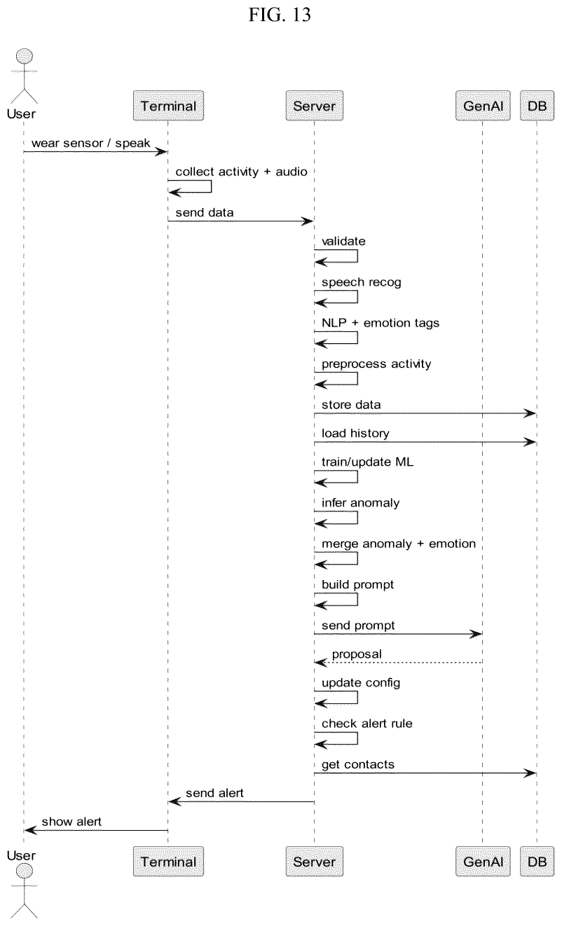

The following describes the processing flow using FIG. 13.

### Step 1

The user wears a wearable sensor and carries the terminal during daily activities.

The input is the user's biological signals and motion, and the output is raw sensor readings (for example, heart rate, acceleration, and step counts) stored in the memory of the wearable sensor.

The wearable sensor periodically samples physical activity information using biosensors and motion sensors, digitizes the readings, and buffers the sampled values with timestamps in its internal storage.

### Step 2

The terminal acquires physical activity information from the wearable sensor.

The input is the buffered sensor readings and timestamps from the wearable sensor, and the output is structured physical activity information stored in the memory of the terminal.

The terminal establishes a wireless connection, such as Bluetooth Low Energy, requests new data frames from the wearable sensor, receives the frames, parses the transmitted bytes into fields such as timestamp, heart rate, and step count, and stores the parsed records in a local data structure.

### Step 3

The terminal records voice information from the user.

The input is the user's spoken utterances near the microphone of the terminal, and the output is digitized audio data saved in an audio buffer.

The terminal samples the microphone signal at a predefined sampling rate, converts the analog sound into digital samples, segments the audio into frames, and stores the segments with associated time information in local storage.

### Step 4

The terminal transmits the physical activity information and voice information to the server.

The input is the locally stored structured physical activity information and digitized audio data, and the output is network messages containing these data delivered to the server.

The terminal packs the data into request payloads, such as JSON objects for the activity data and binary or encoded formats for audio, attaches user identifiers and authentication tokens, opens a secure network connection to the server, and sends the payloads using an application protocol.

### Step 5:

The server receives and validates incoming data from the terminal.

The input is the network messages containing physical activity information and voice information, and the output is validated raw data objects stored in the server memory.

The server accepts the network connection, parses the request headers and bodies, verifies authentication and integrity checks, decodes the payloads into internal data structures, checks the presence and format of required fields, and discards or flags any malformed or unauthorized data.

### Step 6

The server performs speech recognition on the received voice information.

The input is digitized audio data associated with the user, and the output is character information representing recognized text.

The server segments the audio into analysis windows, extracts acoustic features such as mel-frequency cepstral coefficients, feeds feature vectors into an acoustic-linguistic model, decodes the most likely word sequence using a language model, and concatenates recognized tokens into character strings stored in memory.

### Step 7

The server performs natural language processing on the character information to obtain emotion identification information.

The input is the character information corresponding to the user's speech, and the output is emotion information and emotion identification information associated with each text segment.

The server tokenizes the text into words or subword units, applies linguistic analysis such as part-of-speech tagging and dependency parsing, computes sentiment scores and affective features using a trained text analysis model, assigns emotion labels or scores to each segment, and stores the emotion tags together with the original text in a structured format.

### Step 8

The server preprocesses the physical activity information.

The input is the raw structured physical activity records received from the terminal, and the output is cleaned and normalized time-series data.

The server converts timestamps into a unified time zone and standard format, aligns the records onto a regular time grid, detects gaps where measurements are missing, estimates and fills those missing values using interpolation rules, detects outlier values based on statistical thresholds, replaces or flags abnormal points, scales the numeric values into normalized ranges, and constructs time-ordered sequences of heart rate, step count, and activity features.

### Step 9

The server stores the preprocessed physical activity information and emotion identification information in a storage device.

The input is the cleaned time-series data and associated emotion tags, and the output is structured records inserted into database tables indexed by user and time.

The server maps the fields of each record to corresponding columns, such as user identifier, timestamp, heart rate, activity level, data quality flag, emotion label, and emotion score, executes data insertion operations, updates indexes for efficient retrieval, and confirms that the records are durably stored.

### Step 10

The server retrieves historical structured information to learn or update the normal behavior pattern.

The input is a query specifying a user identifier and a historical time range, and the output is a batch of historical physical activity sequences with optional associated emotion tags loaded into the server memory.

The server executes a query on the storage device, collects the returned rows, groups them into contiguous time windows, and converts the grouped records into numeric tensors suitable as training input for the machine learning model.

### Step 11

The server trains or updates the machine learning model representing the normal behavior pattern.

The input is the historical time-series tensors and optional training labels indicating normal segments, and the output is updated model parameters stored in the model representation.

The server initializes the model structure, such as a recurrent or convolutional neural network, defines an error function such as mean squared reconstruction error, computes forward passes on batches of input sequences, calculates gradients of the error with respect to the model weights, updates the weights using an optimization algorithm, and repeats the process over multiple epochs until a stopping criterion is satisfied.

### Step 12

The server applies the trained machine learning model to newly acquired physical activity information.

The input is the latest preprocessed time-series data for the user, and the output is abnormal state information including anomaly scores and abnormality flags.

The server segments the recent data into fixed-length windows, feeds each window into the trained model to obtain outputs such as reconstructions or classification scores, computes anomaly scores based on reconstruction error or probability estimates, compares the anomaly scores with configured thresholds, and labels each window as normal or abnormal while generating a summary abnormality measure over the analysis period.

### Step 13

The server evaluates long-term behavior changes using the abnormal state information and historical structured information.

The input is the current abnormal state information and stored historical records, and the output is updated determination criteria and trend indicators.

The server retrieves previous abnormality statistics, computes trend metrics such as moving averages and slopes, compares recent anomaly patterns against long-term distributions, detects shifts in baseline behavior, and adjusts threshold values or rule parameters to maintain a stable false positive and false negative rate.

### Step 14

The server integrates emotion identification information with the abnormal state information.

The input is time-aligned emotion tags and anomaly scores for the same observation period, and the output is combined feature sets that represent both emotional and physical state.

The server aligns the emotion tags to the corresponding time windows of physical activity data, aggregates the emotion scores over each window, computes joint features such as co-occurrence frequencies of high anomaly scores and negative emotions, and stores these combined features for subsequent analysis and decision-making.

### Step 15

The server generates a prompt sentence for a generative AI model.

The input is the combined feature sets, the current determination criteria, and configuration settings for analysis, and the output is a textual prompt sentence encoding analysis request information.

The server composes a natural-language description of the recent anomaly patterns and emotional states, incorporates summary statistics and current thresholds, embeds explicit questions or requests for optimization, and concatenates these elements into a grammatically coherent text string ready for submission to the generative AI model.

### Step 16

The server transmits the prompt sentence to the generative AI model and receives proposal information.

The input is the prompt sentence generated in the previous step, and the output is proposal information describing suggested threshold changes, feature modifications, or rule updates.

The server sends the prompt sentence to the generative model's interface, waits for the generated text response, parses the response to extract numerical suggestions and rule descriptions, and converts the textual output into an internal representation suitable for configuration updates.

### Step 17

The server generates setting information and updates internal configuration based on the proposal information.

The input is the parsed proposal information, and the output is updated thresholds, rule parameters, and message templates stored in configuration storage.

The server validates the suggested values against safety and stability constraints, merges acceptable suggestions into the existing configuration, modifies parameters such as anomaly thresholds, window sizes, and notification conditions, and writes the new configuration values to persistent storage for use in subsequent processing cycles.

### Step 18

The server determines whether to trigger warning information to a notification target.

The input is the abnormal state information, the integrated emotion information, and the current determination criteria, and the output is a decision flag and, if applicable, prepared notification content.

The server evaluates rule conditions combining anomaly scores, emotional patterns, and long-term trend indicators, checks whether the conditions exceed the dynamically updated criteria, and, when an alert condition is met, selects an appropriate message template and fills in specific details such as the type of abnormality and time period.

### Step 19

The server retrieves contact information and sends the warning information.

The input is the decision flag indicating an alert condition and the prepared notification content, and the output is a transmitted warning message delivered to the notification target.

The server queries the storage device to obtain contact identifiers, such as telephone numbers or electronic mail addresses, formats the warning information according to the communication protocol, invokes a communication interface to send the message, and records delivery identifiers and status codes for logging and audit purposes.

### Step 20:

The terminal receives and presents the warning information to the user or another recipient.

The input is the warning message transmitted by the server, and the output is a displayed or otherwise presented alert on the terminal.

The terminal parses the incoming message, stores it in a local message store, renders the content on a display or outputs it via audio, and provides user interface elements that allow the user or notification target to acknowledge the alert, access additional details, or initiate follow-up actions.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented monitoring systems that process user speech and behavior data typically apply fixed rules or simple statistical thresholds to raw sensor streams or isolated text segments. Such systems suffer from several technical deficiencies. First, they handle heterogeneous data flows (audio signals, textual transcripts, motion data, and temporal context) in a fragmented manner, leading to duplicated processing, inconsistent feature extraction, and inefficient use of computing and storage resources in distributed architectures including terminals and servers. Second, existing systems generally lack an integrated mechanism to generate machine-understandable, semantically rich representations of detected anomalies, so that downstream machine learning components, including generative AI models, must either re-derive context from low-level logs or cannot effectively utilize the data at all. This causes increased latency, increased processor cycles, and higher memory footprint when analyzing patterns over time, particularly in large-scale deployments.

Third, anomaly detection in known systems is usually performed on narrow, single-modality signals and on short time windows, without systematically combining temporal emotion trajectories, linguistic degradation patterns, and behavioral change signals. As a result, the detection accuracy for subtle, early-stage cognitive decline is low, and frequent false positives or false negatives occur, which in turn degrade the reliability of the computer system and require additional manual review. Fourth, current architectures do not provide a standardized way to construct structured prompt sentences for generative AI models from the internal state of detection pipelines. Engineers must hand-code ad hoc prompts that may omit critical contextual features or overload the model with unstructured logs, thereby reducing output quality and increasing token and bandwidth usage between the server and AI services.

Fifth, even when systems attempt to support research use cases, they often lack a built-in, computer-level mechanism for anonymizing, aggregating, and transforming high-volume monitoring data into normalized feature sets and prompt inputs suitable for automated hypothesis generation. This forces human operators to export raw data and manually prepare it with external tools, resulting in fragmented workflows, increased data movement, and elevated privacy risk. Overall, there is a need for an improved computer-implemented technique that (i) unifies multi-modal acquisition and temporal pattern analysis of user voice and behavior, (ii) automatically constructs structured, machine-readable anomaly representations and prompt sentences for generative AI models, and (iii) supports efficient, privacy-preserving reuse of the same processed data for both notification and research tasks, thereby improving the technical functioning of server-side analysis pipelines, memory management, and communication between components.

The specific processing by the specific processing unit 290 of the data processing device 12 in Application Example 2 is realized by the following means.

The present invention provides a server comprising a processor configured to acquire, via a mobile information terminal, voice information and behavior information of a user, to convert the voice information from audio data into text information using an audio processing technique, to convert the behavior information into activity indices based on position information and acceleration information, to analyze an emotional state and linguistic features for the text information using a natural language processing technique, to estimate an emotional state for the voice information based on acoustic feature values, to assign emotion tags and cognitive-function-related features to the text information and store the text information as recorded information, to generate, based on the recorded information and the activity indices, behavior patterns and emotion patterns including temporal transitions, to detect an abnormal pattern in which a cognitive dysfunction is suspected by using statistical processing and machine learning processing, to calculate an abnormality index corresponding to the abnormal pattern, to generate, based on the abnormal pattern and the abnormality index, structured information including an outline of a detected event, recent utterance content, the emotional state, and an activity state, to create a prompt sentence including the structured information for input to a generative artificial intelligence model, to transmit the prompt sentence to the generative artificial intelligence model, to receive response information from the generative artificial intelligence model, and to store the response information as notification information, explanatory information, or anonymized research information. This enables the server to perform integrated, multi-modal feature extraction and temporal pattern detection with reduced redundancy, to automatically transform internal anomaly representations into compact, semantically rich prompt sentences suitable for efficient interaction with a generative AI model, to improve the accuracy and timeliness of cognitive-impairment-related anomaly detection while reducing computational overhead and network usage, and to provide a unified, privacy-preserving data pipeline that supports both real-time notification to related persons and automated research support within a single computer-implemented framework.

The term “mobile information terminal” refers to a portable electronic apparatus having at least a microphone, one or more motion sensors, a communication interface, and a processor, and configured to acquire user voice information and behavior information and to transmit the acquired information to a server.

The term “voice information” refers to information representing sound produced by a user, including at least audio signals obtained by sampling an analog sound waveform with a microphone.

The term “behavior information” refers to information representing physical activity or movement of a user, including at least sensor measurements from a positioning sensor, a motion sensor, or a similar detection element.

The term “audio data” refers to digital data representing sound, obtained by converting analog voice information into a sampled and quantized signal.

The term “text information” refers to character data obtained by converting audio data or other symbolic inputs into a sequence of characters or tokens in a natural language.

The term “audio processing technique” refers to a computational procedure executed by a processor to process audio data, including at least speech recognition that converts audio data into text information.

The term “position information” refers to data indicating a geographic or spatial location of a mobile information terminal or a user, including at least coordinates, area identifiers, or region identifiers.

The term “acceleration information” refers to data output from an acceleration sensor indicating at least one of movement, vibration, or orientation change of a mobile information terminal or a user.

The term “activity indices” refers to numerical or categorical values derived from behavior information, representing activity characteristics such as movement distance, movement area, frequency of movement, or activity level over time.

The term “natural language processing technique” refers to a computational procedure that analyzes text information in a human language, including at least tokenization, syntactic analysis, semantic analysis, or sentiment analysis.

The term “emotional state” refers to a state of user affect such as joy, sadness, anger, anxiety, neutrality, or apathy, inferred from at least one of text information and audio data.

The term “linguistic features” refers to attributes extracted from text information, including at least word frequency, part-of-speech pattern, phrase pattern, repetition pattern, or lexical richness.

The term “acoustic feature values” refers to numerical values derived from audio data, including at least pitch, energy, spectral features, or speech rate, that are used to estimate an emotional state.

The term “emotion tags” refers to labels attached to text information or audio data indicating one or more emotional states of a user, including at least polarity or discrete emotion categories.

The term “cognitive-function-related features” refers to features derived from text information, voice information, or behavior information that are indicative of a user's cognitive performance, including at least confusion expressions, repetition frequency, disorientation phrases, or vocabulary changes.

The term “recorded information” refers to information stored in a memory or storage device, including at least text information, emotion tags, and cognitive-function-related features associated with time information and a user identifier.

The term “behavior patterns” refers to patterns representing temporal changes or tendencies of user behavior, generated from activity indices and recorded information over a period of time.

The term “emotion patterns” refers to patterns representing temporal changes or tendencies of user emotional states, generated from emotion tags or emotional state estimates over a period of time.

The term “temporal transitions” refers to changes of a value, a feature, or a state over time, including at least a sequence, a trend, or a periodicity.

The term “statistical processing” refers to a computational procedure that applies statistical operations to data, including at least aggregation, averaging, variance computation, clustering, or outlier detection.

The term “machine learning processing” refers to a computational procedure that applies a learned model or training algorithm to data, including at least classification, regression, clustering, or anomaly detection using model parameters obtained from training data.

The term “abnormal pattern” refers to a behavior pattern or an emotion pattern that deviates from a reference pattern or normal range, and that is associated with a suspicion of cognitive dysfunction.

The term “cognitive dysfunction” refers to a degradation of cognitive abilities such as memory, attention, orientation, or judgment, inferred from at least user voice information, text information, or behavior information.

The term “abnormality index” refers to a numerical or categorical value representing a degree of abnormality of a detected abnormal pattern, including at least a score, a level, or a probability.

The term “structured information” refers to information arranged in a defined data structure, including at least fields for an outline of a detected event, recent utterance content, emotional state, activity state, and abnormality index.

The term “outline of a detected event” refers to summary data describing a detection result, including at least a type of abnormal pattern, a time of occurrence, and a severity level.

The term “recent utterance content” refers to text information corresponding to one or more user utterances within a predetermined recent time period relative to a detection event.

The term “activity state” refers to information indicating a current or recent activity condition of a user, including at least movement level, movement area, or inactivity duration derived from activity indices.

The term “prompt sentence” refers to a natural-language expression or formatted instruction that includes structured information and that is provided as input to a generative artificial intelligence model to request generation of response information.

The term “generative artificial intelligence model” refers to a computational model that generates output information such as text in response to an input prompt sentence, using parameters learned from training data.

The term “response information” refers to information output from a generative artificial intelligence model in response to a prompt sentence, including at least a notification message, an explanation, a hypothesis, or a recommendation.

The term “notification information” refers to response information selected or formatted for communication to related persons, including at least alerts, warnings, or advice messages.

The term “explanatory information” refers to response information that explains a detected abnormal pattern, an abnormality index, or an underlying reason, and that is intended for a user, a related person, or a professional.

The term “related persons” refers to persons associated with a user, including at least a family member, a care provider, or a medical worker who receives notification information.

The term “communication control function” refers to a function executed by the processor to transmit or receive information via a communication network, including at least message transmission, alarm output, or protocol control.

The term “personal information” refers to information that can identify or is associated with a specific individual, including at least a name, a contact address, a communication identifier, or a direct user identifier.

The term “anonymized information” refers to information obtained by deleting, masking, or replacing personal information in recorded information and activity indices so that an individual cannot be readily identified.

The term “aggregated data” refers to data produced by combining multiple records according to a grouping rule, including at least counts, averages, distributions, or summary statistics.

The term “groups of features” refers to sets of numerical or categorical features derived from anonymized information, used as input to statistical analysis or machine learning processing.

The term “research structured information” refers to structured information that includes aggregated data and groups of features, and that is prepared for use in research analysis or research-supporting prompt sentences.

The term “hypotheses” refers to candidate explanations or conjectures about relationships between features and cognitive dysfunction, generated by a generative artificial intelligence model.

The term “feature proposals” refers to suggestions for new or modified data features or metrics that may improve detection or analysis of cognitive dysfunction, generated by a generative artificial intelligence model.

The term “analysis-method proposals” refers to suggestions for computational methods, models, or procedures for analyzing cognitive-related data, generated by a generative artificial intelligence model.

In the following embodiments, a system including at least one server and at least one terminal is described. The server and the terminal are each implemented by one or more computer devices including at least a processor, a memory, a storage device, and a network interface. The terminal is implemented by a mobile information terminal such as a smartphone or tablet, and the server is implemented by a remote computer system or a cloud-based computer system.

The terminal uses hardware including a built-in microphone, a positioning sensor (for example, a satellite positioning receiver), and a motion sensor (for example, an acceleration sensor and a gyroscope), and uses software including an operating system audio interface, a sensor interface, and a communication stack. The terminal acquires raw analog voice information of a user through the microphone and converts the analog voice information into audio data by using an audio driver and an audio capture library provided by the operating system. The terminal also acquires behavior information by sampling outputs of the positioning sensor and the motion sensor at regular intervals and stores the behavior information in an internal data structure such as a ring buffer or a time-stamped record table.

The terminal converts the behavior information into activity indices by executing numerical computations on the processor. The terminal, for example, computes a movement distance by integrating changes of position information over time, computes a movement area by computing a convex hull or grid coverage of position coordinates, and computes an activity level by summing magnitudes of acceleration vectors over a time window. The terminal represents the activity indices as a structured record including a user identifier, a time stamp, and numerical fields such as “daily_distance”, “nighttime_steps”, and “inactivity_duration”.

The terminal constructs packets that contain the audio data or the already converted text information and the activity indices, and the terminal transmits the packets to the server via a communication network by using a transport protocol such as a secure hypertext transfer protocol. The terminal uses a communication control module that controls packet size, transmission interval, and retry behavior. The terminal thereby reduces network load by aggregating multiple short samples into a single packet when bandwidth is limited, and by adjusting sampling and transmission intervals adaptively based on network conditions.

The server receives packets from multiple terminals through a network interface and temporarily stores the packets in a reception buffer implemented by a message queue or a persistent queue. The server executes a dispatcher module on the processor, and the dispatcher module parses each packet and separates audio data, text information, and activity indices. The server writes raw audio data into a binary object store and registers a pointer to the binary object in a relational table. The server writes activity indices and text information into a normalized database schema including tables for “Utterance”, “EmotionFeature”, and “ActivitySummary”, with foreign keys linking them via a user identifier and a time stamp.

The server converts audio data into text information by using an automatic speech recognition module. In one embodiment, the server uses an external speech recognition service accessible through an application programming interface. The server sends audio data in a compressed format to the speech recognition service and receives a set of tokens, word-level time stamps, and confidence scores. The server then executes a text normalization module that performs language-specific normalization such as punctuation restoration, script conversion, and token merging. The server stores the normalized text into the “Utterance” table, together with a reference to the original audio data.

The server computes acoustic feature values directly from the audio data by using a digital signal processing library. The server, for example, computes a short-time Fourier transform, extracts mel-frequency cepstral coefficients, pitch, formant frequencies, and frame-level energy, and stores these features in a compact vector representation. The server compresses sequences of frame-level features by applying pooling operations such as mean pooling and max pooling over time windows, thereby reducing memory usage and improving computational efficiency for subsequent machine learning processing.

The server applies a natural language processing module to the text information in order to derive linguistic features and to estimate an emotional state. The server uses tokenization, part-of-speech tagging, dependency parsing, and named-entity recognition to compute counts of part-of-speech tags, syntactic pattern frequencies, and vocabulary richness measures such as type-token ratio. The server detects specific phrase templates associated with confusion, disorientation, or repetition by matching token sequences and by computing similarity to stored phrase patterns using vector representations. The server also applies a sentiment analysis model to obtain scores for affect dimensions such as valence and arousal.

The server estimates an emotional state from the acoustic feature values by using a trained neural-network-based classifier. In one embodiment, the server uses a neural network including at least one convolutional layer that processes spectrogram-like feature maps, followed by at least one recurrent layer that processes temporal sequences, and at least one fully connected layer that outputs probabilities for emotion categories. The server trains this neural network offline by using a training dataset that contains audio samples labeled with emotion categories. The server uses a loss function such as cross-entropy loss and updates network weights by using stochastic gradient descent or a similar optimization method. During training, the server may use data augmentation methods such as time stretching, pitch shifting, and additive noise to increase robustness to real-world conditions.

The server fuses text-based emotion information and acoustic-based emotion information into a single emotion tag and emotion feature vector. The server, for example, concatenates sentiment scores, emotion category probabilities derived from text, and emotion category probabilities derived from audio, and then applies a small feed-forward neural network or a logistic regression model to compute a final emotion tag and a confidence score. The server stores the final emotion tag and the emotion feature vector in the “EmotionFeature” table, linked to the corresponding utterance.

The server generates behavior patterns and emotion patterns by aggregating activity indices and emotion feature vectors over time. The server uses a sliding time window to compute summary values such as daily counts of confusion-related utterances, average emotion scores per day, trend slopes of positive versus negative emotion, and changes in movement radius compared with historical baselines. The server stores the aggregated values in a “PatternSummary” structure for each user and time period.

The server detects abnormal patterns associated with cognitive dysfunction by applying machine learning processing to the “PatternSummary” data. In one embodiment, the server uses a recurrent neural network model, such as a long short-term memory network, that takes as input a sequence of feature vectors representing consecutive time windows. The server trains this model on a dataset where sequences are labeled as normal or as containing cognitive impairment signals. The server uses a loss function such as binary cross-entropy and backpropagation through time to update the network weights. In another embodiment, the server uses an unsupervised anomaly detection algorithm such as an autoencoder. The server trains the autoencoder on normal sequences and measures reconstruction error; sequences with reconstruction error exceeding a threshold are treated as abnormal patterns.

The server computes an abnormality index for each user and each time period. The server combines outputs of multiple models, such as a probability from the classification model, a reconstruction error from the autoencoder, and selected rule-based scores, into a single scalar index by using a weighted summation or a learned meta-model. The server stores the abnormality index in the “PatternSummary” table and compares the abnormality index against a threshold that may be user-specific or population-specific.

The server generates structured information describing a detected abnormal pattern. The server constructs a record that includes at least the abnormality index, a label of abnormal pattern type, a compressed representation of recent utterance content, condensed emotion trajectories, and recent activity indices. The server encodes this structured information as a compact textual or key-value format designed specifically for efficient use as input to a generative AI model. The server thereby reduces redundant information and improves the relevance of inputs to the generative AI model, which contributes to reduced token usage and processing time on the generative AI model.

The server constructs a prompt sentence for the generative AI model by combining fixed instruction templates with the structured information. The server uses rule-based formatting that is different from simple natural language summarization. The server, for example, inserts the structured information into designated placeholders in a template that explicitly instructs the generative AI model about the desired output format and tone. The server may use, as examples of a prompt sentence, “Analyze the following sequence of emotion-tagged utterances from an elderly user and explain whether they may indicate early-stage cognitive decline. Suggest what kind of medical consultation should be recommended.” or “Given a user whose confusion-related statements increased from 2 per week to 15 per week over a month, generate a reassuring but clear message to the family encouraging them to arrange a cognitive assessment.”

The server transmits the prompt sentence and, if necessary, additional structured information to the generative AI model through an application programming interface. The server configures parameters such as maximum output length, sampling temperature, and top-k or top-p sampling thresholds to generate stable, predictable outputs suitable for medical-style notifications. The server receives response information from the generative AI model and performs validation processing that checks for prohibited phrases, excessive length, or missing required elements. The server applies post-processing steps such as truncation, insertion of disclaimers, and localization of wording when necessary.

The server stores the validated response information in association with the abnormal pattern that triggered generation of the prompt sentence. The server writes the response information into a “NotificationHistory” table that includes fields such as user identifier, event time, abnormality index, notification text, and communication result. The server ensures that the response information is accessible both to a notification module and to a research module.

The server performs notification processing by transmitting messages to related persons. The server retrieves contact information of related persons, such as telephone numbers and electronic addresses, from a “ContactProfile” table. The server uses a communication control function that wraps lower-level protocols and external message delivery services. The server sends text notifications or alarm signals to related persons only when the abnormality index exceeds a configured threshold and when further policy conditions are satisfied, such as a minimum time interval since the last notification.

The server generates anonymized information for research. The server removes direct identifiers such as names and direct contact information from recorded information and activity indices and replaces them with random identifiers. The server aggregates anonymized information into summary records that represent groups of users or time ranges. The server computes groups of features including average emotion trends, distribution of confusion phrases, and trajectories of activity indices before and after detection of abnormal patterns. The server stores these aggregated data and feature groups in a “ResearchDataset” structure.

The server constructs research-supporting prompt sentences for the generative AI model by using the “ResearchDataset” structure. The server, for example, creates a prompt sentence such as “Using the attached de-identified dataset summary of emotion and language features before dementia diagnosis, propose new hypotheses about early behavioral markers of cognitive dysfunction.” or “Suggest feature-engineering strategies for detecting early cognitive decline from daily conversation transcripts and emotion tags in elderly users.” The server transmits these prompt sentences and selected aggregated statistics to the generative AI model and receives hypotheses, feature proposals, or analysis-method proposals. The server stores such proposals together with the underlying data context, enabling researchers to test and refine new model architectures and feature sets.

The server improves computer technology in several concrete ways by using these specific data structures and processing flows. The server reduces overall processing load by performing feature extraction and aggregation at the server side and by using dimension-reduced feature vectors as inputs to machine learning models, rather than passing raw audio streams and full text logs repeatedly through the models. The server improves detection accuracy by combining multi-modal features from audio, text, and activity sensors and by modeling temporal transitions using sequence models. The server reduces communication load by transmitting structured summaries rather than raw logs to the generative AI model and by adaptively controlling packetization and sampling at the terminal.

The server and the terminal cooperatively enable functions that are not achievable by manual human observation or simple digitization of paper processes. The terminal performs real-time feature preprocessing and adaptive transmission control in accordance with sensor conditions and network status, and the server executes non-conventional anomaly detection pipelines that use autoencoders and recurrent models trained on large-scale time-series feature representations. The system thereby performs complex pattern recognition and context-aware prompt construction according to rules and models that differ from human heuristic analysis.

Alternative embodiments are also possible. The terminal may execute some or all of the speech recognition and emotion analysis locally by using a compressed neural network running on a mobile inference framework, and may transmit only text information and emotion tags to the server. The server may replace the recurrent neural network with a temporal convolutional network or a transformer-based sequence model for pattern detection. The generative AI model may be hosted within the same server environment instead of being accessed as an external service, and may be fine-tuned on a corpus of domain-specific notifications. The server may employ different anonymization strategies, such as k-anonymity or differential-privacy-based noise injection, when generating research datasets.

The user interacts with the system through the terminal. The user may view summaries of recent emotional states and activity patterns displayed by the terminal and may send queries in the form of free-text prompt sentences to the server. The user, for example, may input “Explain in simple terms what kind of behavior pattern was detected and what we should do next week,” and the server may append relevant detection results to this prompt sentence, send the combined prompt to the generative AI model, and deliver a customized explanation and action plan to the terminal. The terminal displays graphs, timelines, and generated explanations on a graphical interface and may provide interaction controls that allow the user to filter events, adjust sensitivity, or change notification preferences.

By integrating the above-described acquisition, feature extraction, pattern detection, prompt construction, and generative AI interaction in a single technical framework, the server and the terminal cooperate to provide a computer-implemented technique that improves internal data management, processing efficiency, and communication efficiency of multi-modal monitoring systems. The described embodiments thereby support practical early detection of cognitive dysfunction and provide a basis for further technical improvements in computer-assisted health monitoring and research.

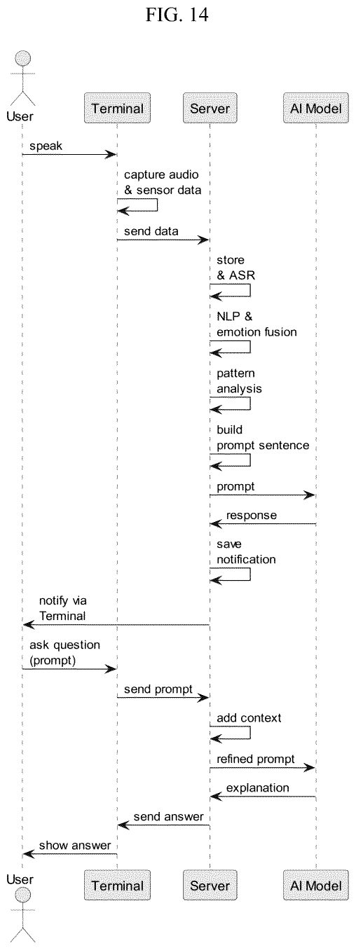

The following describes the processing flow using FIG. 14.

### Step 1

The user speaks near the terminal in daily life.

Input: Human speech (airborne sound waves).

Output: Analog voice signal at the microphone.

The terminal receives the sound waves with a built-in microphone and converts the analog voice signal into an electrical signal, which is then sampled and quantized by an audio codec on the terminal hardware.

### Step 2

The terminal acquires digital audio data from the audio codec.

Input: Analog electrical signal from the microphone.

Output: Digital audio frames (for example, 16-bit PCM at 16 kHz).

The terminal uses an operating-system audio API to read successive buffers of audio samples, attaches a user identifier and timestamps to each buffer, and stores the audio frames in a temporary in-memory buffer.

### Step 3

The terminal acquires behavior information from motion and position sensors.

Input: Raw sensor readings from an accelerometer, gyroscope, and positioning module.

Output: Time-stamped behavior records.

The terminal periodically reads acceleration vectors and location coordinates, converts sensor readings into a normalized coordinate system, and stores each reading with a timestamp in a local data structure such as a ring buffer or small local database.

### Step 4

The terminal computes activity indices from the behavior information.

Input: Time-stamped behavior records for a defined time window.

Output: Activity indices such as movement distance, movement radius, and activity level.

The terminal calculates movement distance by summing distances between consecutive position samples, calculates movement radius by determining the maximum distance from a reference location, and calculates an activity level by integrating the magnitude of acceleration over the time window; the terminal writes these computed scalar values into a compact activity-index record.

### Step 5

The terminal packages audio data and activity indices into a transmission payload.

Input: Buffered audio frames, activity indices, user identifier, and timestamps.

Output: Encoded payload object ready for network transmission.

The terminal constructs a data structure that includes metadata (user ID, device ID, time range) and binary or encoded audio segments together with associated activity indices, serializes the structure into a compact format, and queues it in an outgoing transmission buffer.

### Step 6

The terminal transmits the payload to the server over a network.

Input: Encoded payload object from the outgoing transmission buffer.

Output: Network packets sent via a secure communication channel.

The terminal opens a secure connection, splits the payload into packets according to the transport protocol, adds necessary headers (destination, authentication token), and sends the packets to the server, retrying transmission if an error occurs.

### Step 7

The server receives and parses the payload from the terminal.

Input: Network packets containing the encoded payload.

Output: Separated audio data, activity indices, and metadata.

The server reassembles the packets into the original payload, verifies authentication data, deserializes the payload structure, and extracts the audio segments, the activity indices, the user identifier, and the timestamps into separate internal variables.

### Step 8

The server stores raw data into persistent storage.

Input: Extracted audio segments, activity indices, user identifier, and timestamps.

Output: Database and object-store entries referencing the raw data.

The server writes audio segments as binary objects into a file store or object store, writes pointers to those objects into an “Utterance” table, and writes activity indices into an “ActivitySummary” table, all indexed by the user identifier and time.

### Step 9

The server converts audio data into text information using speech recognition.

Input: Audio segments and associated metadata from the “Utterance” table.

Output: Text transcripts with word-level timestamps and confidence scores.

The server sends audio segments to a speech recognition engine with language and acoustic settings, receives a sequence of recognized tokens and word boundaries, and performs text normalization (such as punctuation insertion and character normalization) to produce a clean transcript string for each audio segment.

### Step 10

The server computes acoustic feature values from the audio data.

Input: Audio segments for which transcripts exist.

Output: Acoustic feature vectors representing emotional and prosodic characteristics.

The server applies signal-processing operations such as framing, windowing, and Fourier transforms, computes features including pitch, energy, spectral coefficients, and speech rate, and aggregates frame-level values into a fixed-length feature vector for each utterance.

### Step 11

The server analyzes the text information using natural language processing.

Input: Normalized text transcripts from the speech recognition output.

Output: Linguistic feature vectors and preliminary emotion scores.

The server tokenizes each transcript, assigns part-of-speech tags, detects key phrase patterns associated with confusion or repetition, computes statistics such as vocabulary richness, and applies a text-based sentiment or emotion classifier to generate scores for various emotion categories.

### Step 12

The server estimates an emotional state from acoustic feature values.

Input: Acoustic feature vectors for each utterance.

Output: Category probabilities and scores for acoustic-based emotions.

The server feeds each acoustic feature vector into a trained neural network model that outputs probabilities for different emotional categories, interprets the probabilities as acoustic emotion scores, and stores these scores as part of an “EmotionFeature” record.

### Step 13

The server fuses text-based and acoustic-based emotion results.

Input: Text-based emotion scores and acoustic-based emotion scores for the same utterance.

Output: Final emotion tag and combined emotion feature vector.

The server concatenates text-based and acoustic-based scores, applies a fusion model or rule set to compute a single dominant emotion category and an overall confidence value, labels the utterance with this final emotion tag, and stores the combined feature vector and tag.

### Step 14

The server aggregates emotion features and activity indices over time.

Input: Sequences of emotion feature vectors and activity indices for a user across multiple time windows.

Output: Pattern summary records describing behavior and emotion patterns.

The server groups data by fixed or adaptive time windows, computes per-window statistics such as counts of confusion-related utterances, average emotion scores, and changes in activity level, and writes these aggregated values into a “PatternSummary” structure per user and time window.

### Step 15

The server detects abnormal patterns associated with cognitive dysfunction.

Input: Pattern summary records over a historical time range for a user.

Output: Detection results indicating normal or abnormal patterns with scores.

The server passes sequences of pattern summary vectors through a sequence model or anomaly-detection algorithm, computes an abnormality index that quantifies deviation from typical patterns, compares the index with a reference threshold, and marks a pattern as abnormal if the threshold is exceeded.

### Step 16

The server generates structured information describing a detected abnormal pattern.

Input: Detection results, recent utterances, emotion tags, and activity indices.

Output: A structured information record summarizing the abnormal event.

The server collects the abnormality index, pattern type, short excerpts of recent transcripts, recent emotion trajectories, and recent activity indices, arranges these fields into a defined structure, and stores the structure as a compact summary object.

### Step 17

The server constructs a prompt sentence for a generative AI model.

Input: The structured information record for an abnormal event.

Output: A natural-language prompt sentence containing embedded structured data.

The server selects a template appropriate for the target output (for example, family notification or researcher support), fills template slots with elements from the structured information, and produces a prompt sentence such as: “Analyze the following recent emotion-tagged utterances and activity summary for an elderly user and write a concise, non-alarming message for the user's family explaining that possible early cognitive decline has been detected and recommending that they arrange a medical consultation.”

### Step 18

The server sends the prompt sentence to the generative AI model and receives response information.

Input: Prompt sentence, model configuration parameters, and optionally additional structured information.

Output: Generated text response containing notification or explanatory content.

The server packages the prompt sentence and configuration (maximum length, sampling settings) into an API request, transmits the request to the generative AI model endpoint, waits for completion, and parses the returned text as response information.

### Step 19

The server validates and stores the generated response information.

Input: Generated text response from the generative AI model.

Output: Validated notification or explanatory text stored in persistent storage.

The server checks the response for required elements (for example, explanation and recommendation), verifies that forbidden expressions are not present, applies any necessary truncation or formatting, and writes the validated text into a “NotificationHistory” or “ExplanationHistory” record linked to the corresponding abnormal event.

### Step 20

The server determines whether to notify related persons based on the abnormality index and policy rules.

Input: Detection results, abnormality index, and notification history for the user.

Output: Decision flag indicating whether to send a notification.

The server compares the abnormality index with configured thresholds, checks cooldown intervals and user preferences, and sets a flag to “notify” or “do not notify” by evaluating these conditions.

### Step 21

The server sends notification messages to related persons when a notification is required.

Input: Validated notification text, contact information of related persons, and decision flag.

Output: Sent messages and logged delivery results.

The server retrieves contact addresses, chooses an appropriate communication channel (such as text message or email), formats the notification text for the channel, sends messages through a communication interface, and logs the status of each sent message in association with the abnormal event.

### Step 22

The terminal receives and displays notifications for the user or caregiver.

Input: Notification messages addressed to the terminal or push-notification payloads.

Output: Visual or audible presentation of notification content on the terminal.

The terminal receives notifications through a push-notification service or direct message channel, displays the text and key values (such as type of pattern and recommended action) in a user interface, and optionally plays alert sounds or vibration to attract attention.

### Step 23

The user reviews the notification and optionally submits a follow-up query as a prompt sentence.

Input: Displayed notification text and user interaction (touch or voice).

Output: A user-generated prompt sentence requesting further explanation or advice.

The user reads the summary and may enter a query such as “Explain in simple terms what kind of behavior pattern was detected and what we should do next week,” using a text input field or voice dictation; the terminal converts any voice input into text and stores the query as a prompt sentence.

### Step 24

The terminal sends the user-generated prompt sentence to the server.

Input: Prompt sentence and context identifiers (user ID, relevant abnormal event ID).

Output: An API request containing the prompt sentence and context.

The terminal constructs a request body that includes the prompt sentence and identifiers of the related detection event, encrypts the request, and transmits it to the server over the network.

### Step 25

The server augments the user-generated prompt sentence with technical context and queries the generative AI model.

Input: User-generated prompt sentence, detection results, and structured information.

Output: A refined prompt sentence and a new generated response from the generative AI model.

The server appends concise context (such as abnormality index and summarized patterns) to the user's prompt sentence, builds a combined prompt instructing the generative AI model to provide detailed explanation or an action plan, sends the prompt to the generative AI model, and receives an extended response.

### Step 26

The server returns the extended response to the terminal for user display.

Input: Extended explanation or action-plan text generated by the generative AI model.

Output: Response payload ready for display.

The server formats the extended response, attaches identifiers to associate it with the original query, and sends the response back to the terminal using an application programming interface.

### Step 27

The terminal displays the extended explanation and recommendations to the user.

Input: Response payload from the server containing extended explanation or action plan.

Output: Updated user interface view presenting detailed information.

The terminal renders the text in a readable layout, may highlight key recommendations such as “schedule a consultation” or “monitor sleep,” and provides scrolling and navigation controls so the user or caregiver can review the information.

### Step 28:

The server periodically generates anonymized datasets and research-support prompt sentences.

Input: Accumulated recorded information, activity indices, detection results, and notification history.

Output: Anonymized research datasets and research-oriented prompt sentences.

The server removes personal identifiers, aggregates features across users and time, composes research-support prompts such as “Using the following aggregated feature patterns that precede dementia diagnosis, propose new behavioral and linguistic markers,” and uses these prompts with the generative AI model to obtain hypotheses and feature proposals for research.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

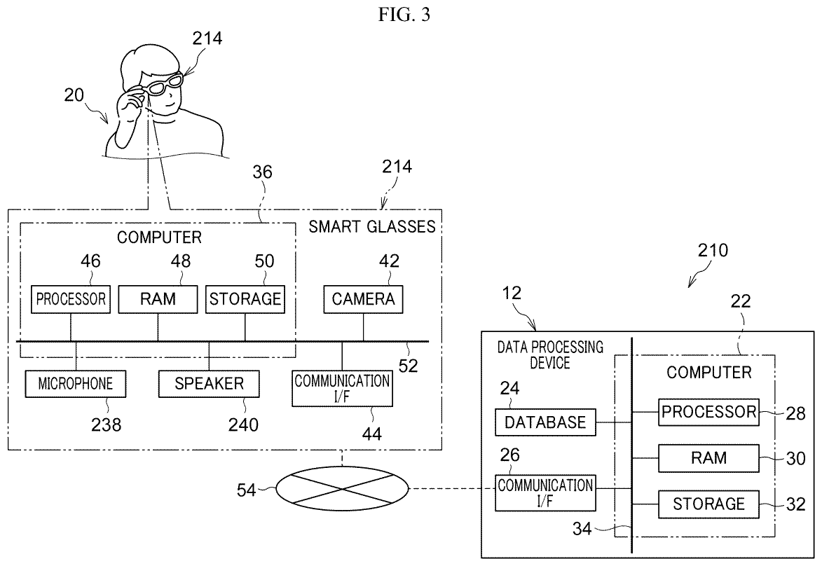

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

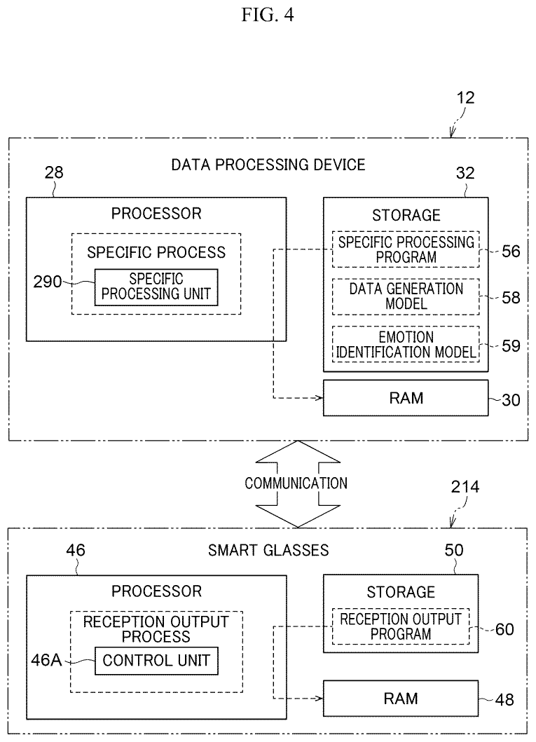

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https: //openai. com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

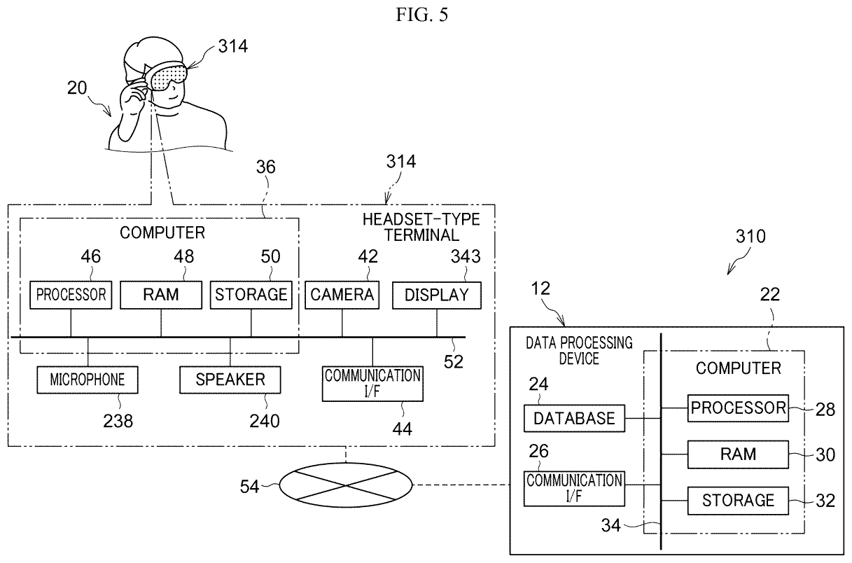

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

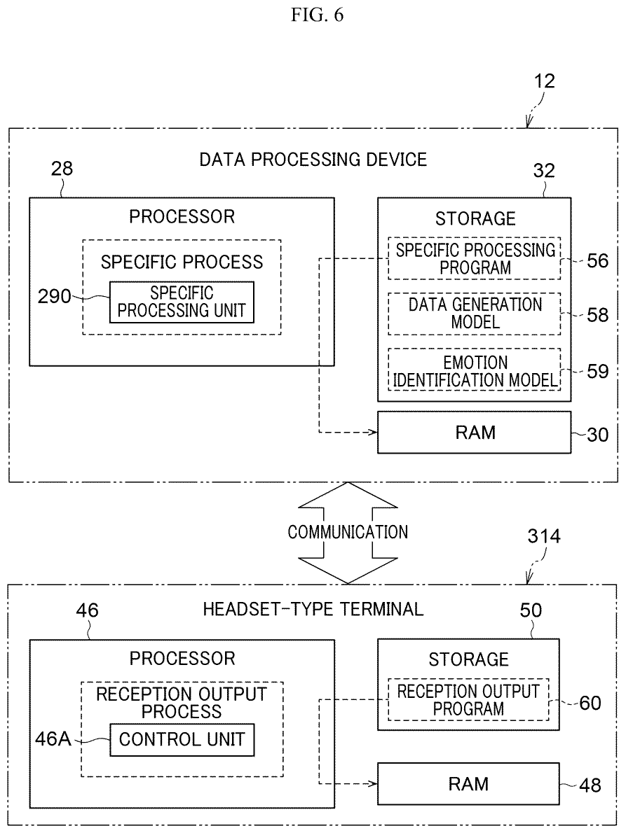

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https: //openai. com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

Fourth Exemplary Embodiment

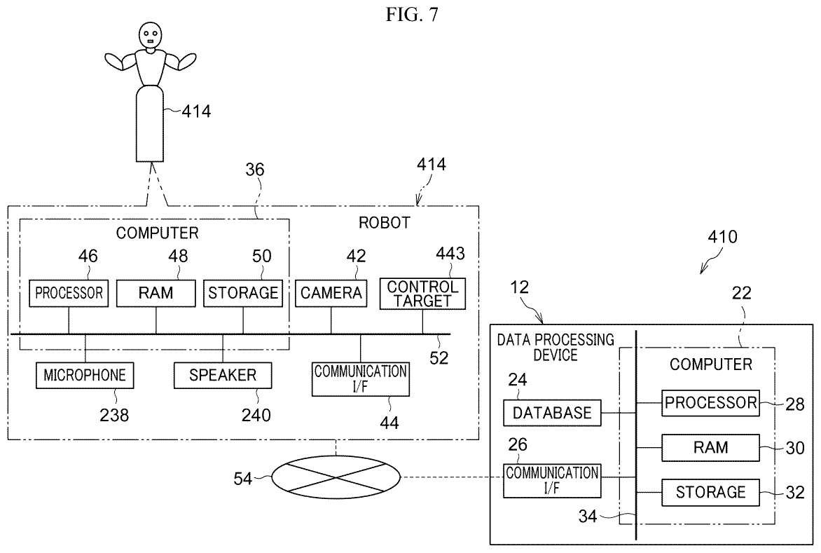

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

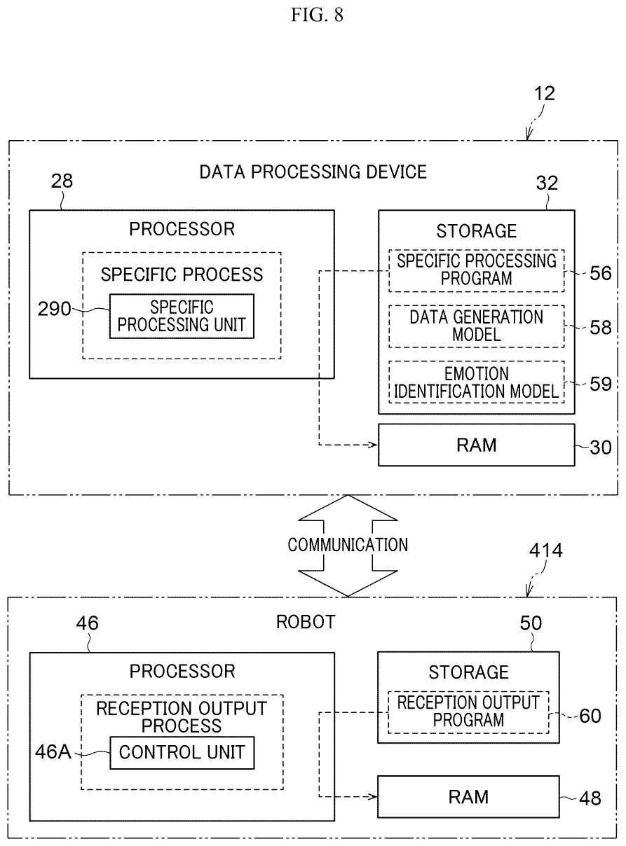

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https: //openai. com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

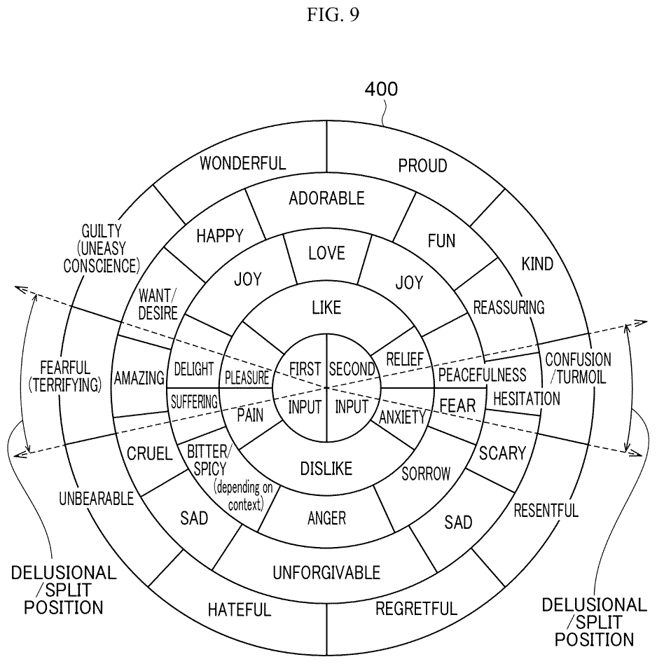

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

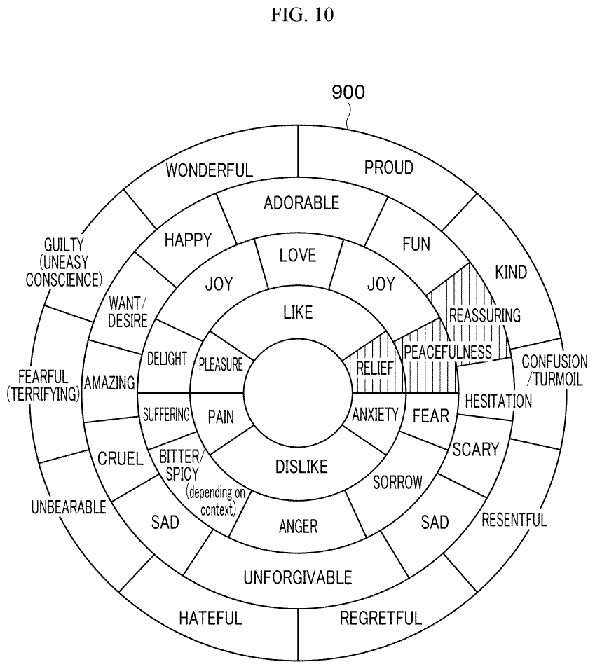

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire a voice signal of a user by controlling an acoustic input unit and an operation control application of a portable terminal device to collect the voice signal and to transmit the voice signal to a speech recognition processing unit to convert the voice signal into character information,
execute emotion analysis processing on the character information by using a natural language processing program to generate emotion-annotated character information to which at least one emotion label indicating a positive state, a negative state, or a neutral state and an emotion score are attached,
store the emotion-annotated character information, user identification information associated with the emotion-annotated character information, and time information associated with the emotion-annotated character information in a data management storage device,
generate, based on a set of the emotion-annotated character information accumulated in the data management storage device, feature information representing a behavioral tendency suspected of being related to cognitive dysfunction by using a machine learning processing program, construct a classification model or a discrimination model that estimates presence or degree of suspicion of the cognitive dysfunction by using the feature information as input, and detect, by using the classification model or the discrimination model, a behavioral pattern indicating a sign of the cognitive dysfunction for each user,
generate, based on a detection result of the behavioral pattern and statistical information of the emotion-annotated character information, a prompt sentence for obtaining a proposal regarding at least one of an analysis policy for the sign of the cognitive dysfunction, a feature design, a threshold setting, and a notification condition setting, and cause the prompt sentence to be input to a generative information processing model, and
update, based on response information obtained from the generative information processing model, processing content or a parameter of at least one of the emotion analysis processing and the behavioral pattern detection processing.

### (Supplementary 2)

The system according to supplementary 1,
wherein the processor is configured to
execute a notification process, when an estimated degree of suspicion of the cognitive dysfunction obtained by the classification model or the discrimination model exceeds a predetermined determination criterion, by referring to user information stored in the data management storage device and transmitting, via an electronic communication network, alert information to at least one of a guardian, a healthcare professional, and the user.

### (Supplementary 3)

The system according to supplementary 1,
wherein the processor is configured to
execute a statistical process on the emotion-annotated character information stored in the data management storage device and on a detection result of the behavioral pattern, the statistical process analyzing at least one of a change tendency of the emotion label over time, an appearance frequency of a specific phrase, a distribution change of the emotion score, and a transition of an output value of the classification model or the discrimination model, and use an analysis result of the statistical process for generating an additional prompt sentence to be input to the generative information processing model.

### Application Example 1

### (supplementary 1)

A system comprising a processor and a memory storing instructions,
wherein the processor is configured to execute the instructions to acquire voice information of a user by using a mobile information processing terminal and store the voice information in a temporary storage region,
receive, via a communication network, the voice information transmitted from the mobile information processing terminal, and convert the voice information into character information by using a speech recognition processing resource,
perform emotion analysis on the character information by using a natural language processing resource and attach, to the character information, an emotion tag indicating an emotion type and an emotion intensity,
store, in a data storage device, the character information with the attached emotion tag and corresponding time information,
calculate, on the basis of a plurality of items of emotion-tagged character information stored in the data storage device, at least one of an occurrence frequency of the emotion tags, a temporal change of the emotion tags, and a linguistic feature of an expression, and generate a behavioral-pattern determination feature vector as a feature vector representing the calculated result,
input the behavioral-pattern determination feature vector into a prediction model configured by a machine learning processing resource, and calculate a presence or absence of a behavioral pattern related to cognitive function decline and a risk index representing the behavioral pattern,
generate, on the basis of the risk index and the emotion-tagged character information, a prompt sentence to be used as an input to a generative information processing model, and instruct the generative information processing model to perform analysis and generate an explanatory text regarding a sign of dementia, and
when the risk index calculated by the prediction model exceeds a predetermined threshold, transmit notification information including at least one of the analysis and the explanatory text to a related-person terminal or a user terminal.

### (Supplementary 2)

The system according to supplementary 1,
wherein the processor is configured to execute the instructions to
retrain the prediction model on the basis of past emotion-tagged character information stored in the data storage device and feedback information acquired from the user, and update a correspondence between the behavioral-pattern determination feature vector and the risk index.

### (Supplementary 3)

The system according to supplementary 1,
wherein the processor is configured to execute the instructions to cause the generative information processing model, on the basis of the prompt sentence and the behavioral-pattern determination feature vector, to generate a report text summarizing at least one of a transition of the emotion tags, a frequency of utterances related to forgetfulness, and a tendency of linguistic expressions indicative of cognitive function decline, and present the report text, for evaluation by a medical worker or a caregiver, on the related-person terminal.

### Example 2

### (Supplementary 1)

A system comprising a processor,
wherein the processor is configured to
acquire, via a mobile terminal connected to an information processing apparatus, voice information and physical activity information of a user,
convert the voice information into character information by performing a speech recognition process, apply natural language processing to the character information to extract emotion information, and assign emotion identification information based on the emotion information, perform numerical processing and preprocessing on the physical activity information, the preprocessing including at least unifying time information, complementing missing values, correcting outliers, and normalizing numerical values,
store the preprocessed physical activity information as structured information in an information storage apparatus,
use a machine learning model on the structured information to learn a normal behavior pattern of the user and calculate abnormal state information according to a degree of deviation from the normal behavior pattern,
generate a prompt sentence including analysis request information for analyzing a sign of cognitive function decline on the basis of the emotion identification information and the abnormal state information, and instruct a generative information processing model to receive the prompt sentence as input, and
when the abnormal state information satisfies a predetermined determination criterion, acquire contact information of a notification target and transmit warning information to the contact information.

### (Supplementary 2)

The system according to supplementary 1,
wherein the processor is configured to
evaluate a long-term change in a behavior pattern of the user by comparing the abnormal state information calculated by the machine learning model with past structured information stored in the information storage apparatus, and dynamically update the predetermined determination criterion based on a result of the evaluation.

### (Supplementary 3)

The system according to supplementary 1,
wherein the processor is configured to
generate setting information for changing at least one of content of the analysis request information and content of the warning information based on proposal information output from the generative information processing model, and automatically adjust at least one of a threshold of the machine learning model and a notification content according to the setting information.

### Application Example 2

### (Supplementary 1)

A system comprising a processor,
wherein the processor is configured to
acquire, via a mobile information terminal, voice information and behavior information of a user, convert the voice information from audio data into text information by using an audio processing technique, and convert the behavior information into activity indices based on position information and acceleration information,
analyze an emotional state and linguistic features for the text information by using a natural language processing technique, estimate an emotional state for the voice information based on acoustic feature values, and assign, based on analysis results, emotion tags and cognitive-function-related features to the text information and store the text information as recorded information,
generate, based on the recorded information and the activity indices, behavior patterns and emotion patterns including temporal transitions, detect an abnormal pattern in which a cognitive dysfunction is suspected by using statistical processing and machine learning processing, and calculate an abnormality index corresponding to the abnormal pattern,
generate, based on the abnormal pattern and the abnormality index, structured information including an outline of a detected event, recent utterance content, the emotional state, and an activity state, and create a prompt sentence including the structured information for input to a generative artificial intelligence model,
and transmit the prompt sentence to the generative artificial intelligence model and store response information obtained from the generative artificial intelligence model as notification information or explanatory information.

### (Supplementary 2)

The system according to supplementary 1,
wherein the processor is configured to
generate, when the abnormal pattern and the abnormality index are determined to exceed a predetermined reference value, notification content for related persons including at least one of a family member, a care provider, and a medical worker based on the response information and the structured information, and perform message transmission or alarm output to the related persons by using a communication control function.

### (Supplementary 3)

The system according to supplementary 1,
wherein the processor is configured to
generate anonymized information by deleting or replacing personal information from the recorded information and the activity indices, create aggregated data and groups of features based on the anonymized information, generate a research-supporting prompt sentence for the generative artificial intelligence model by using research structured information including the aggregated data and the groups of features, and obtain at least one of hypotheses, feature proposals, and analysis-method proposals concerning early detection and measures of cognitive dysfunction from the generative artificial intelligence model.

What is claimed is:

1. A system comprising:
circuitry configured to:
receive, via a communication interface coupled to a packet-switched network, audio-derived text data and associated user identification data and time information from a terminal device;
execute affective state analysis processing on the text data using a natural language processing model to generate affective-annotated text data comprising at least one affective label and an affective score, and store the affective-annotated text data in a storage device in association with the user identification data and the time information;
generate, from a set of stored affective-annotated text data, feature information representing behavioral tendency patterns, construct a classification model that estimates a presence or degree of deviation in the behavioral tendency patterns based on the feature information, and execute behavioral pattern detection for each user using the classification model;
compute statistical information over time for the affective labels, affective scores, phrase occurrences, and classification model outputs, generate a prompt sentence based on the behavioral pattern detection results and the statistical information, execute inference processing using a generative neural network model with the prompt sentence as input, and receive response information from the generative neural network model; and
update at least one of parameters, processing content, and configuration of the affective state analysis processing and the behavioral pattern detection processing based on the response information.

2. The system according to claim 1, wherein the circuitry is configured to receive the audio-derived text data by controlling a speech recognition model to convert an audio signal received from the terminal device into text.

3. The system according to claim 2, wherein the circuitry is configured to generate the affective-annotated text data by assigning at least one of a positive label, a negative label, and a neutral label to text segments based on sentiment scores computed by the natural language processing model.

4. The system according to claim 3, wherein the circuitry is configured to segment the text data into analysis units based on at least one of a temporal window and a phrase boundary, and apply the affective state analysis processing to each analysis unit independently.

5. The system according to claim 4, wherein the circuitry is configured to generate the feature information by extracting at least one of affective label frequency distributions, affective score variance metrics, and phrase occurrence patterns from the stored affective-annotated text data.

6. The system according to claim 5, wherein the circuitry is configured to construct the classification model by applying a machine learning training procedure to the feature information, using labeled training instances to train a discrimination boundary that separates normal behavioral tendency patterns from anomalous behavioral tendency patterns.

7. The system according to claim 1, wherein the circuitry is configured to compute statistical information comprising at least one of moving average values, trend metrics, and deviation metrics computed over a time window applied to the affective scores and classification model outputs.

8. The system according to claim 7, wherein the circuitry is configured to generate the prompt sentence by embedding the statistical information, the behavioral pattern detection results, and at least one of an analysis policy parameter and a threshold configuration parameter as input fields for the generative neural network model.

9. The system according to claim 8, wherein the circuitry is configured to update the threshold configuration parameter of the classification model based on the response information received from the generative neural network model.

10. The system according to claim 1, wherein the circuitry is configured to detect a behavioral pattern satisfying a notification condition based on the classification model output, generate a notification message in response to the detection, and transmit the notification message to a designated terminal device via the communication interface.

11. The system according to claim 10, wherein the circuitry is configured to generate the notification message by constructing a summary of the behavioral pattern detection results and the statistical information, and formatting the summary as the notification message for transmission.

12. The system according to claim 1, wherein the circuitry is configured to update model parameters of the natural language processing model based on accumulated affective-annotated text data, to adapt affective state analysis to changing text patterns over time.

13. The system according to claim 12, wherein the circuitry is configured to perform incremental learning on the classification model using newly accumulated affective-annotated text data to update the discrimination boundary.

14. The system according to claim 1, wherein the circuitry is configured to store the prompt sentence and the response information as record information in the storage device, and update generation conditions for subsequent prompt sentences based on the stored record information.

15. The system according to claim 14, wherein the circuitry is configured to retrieve, from the stored record information, prior response information associated with similar statistical information using a similarity search, and incorporate the retrieved information as context in a subsequent prompt sentence.

16. The system according to claim 1, wherein the circuitry is configured to generate visualization data from the statistical information and transmit the visualization data to the terminal device for display as a time-series trend representation.

17. The system according to claim 16, wherein the circuitry is configured to include detected anomalous behavioral pattern indicators in the visualization data, and update the visualization data in response to updated statistical information.

18. A system comprising:
circuitry configured to:
receive, via a communication interface coupled to a packet-switched network, audio-derived text data and user identification data from a terminal device, execute affective state analysis using a natural language processing model to generate affective-annotated text data comprising affective labels and affective scores, and store the affective-annotated text data in a storage device;
generate feature information representing behavioral tendency patterns from the stored affective-annotated text data, construct a classification model that estimates a deviation degree based on the feature information, and execute behavioral pattern detection using the classification model;
compute statistical information comprising at least one of trend metrics and deviation metrics over a time window, generate a prompt sentence embedding the behavioral pattern detection results, the statistical information, and a threshold configuration parameter, and execute inference processing using a generative neural network model to obtain response information; and
update at least one of the threshold configuration parameter of the classification model, the affective state analysis processing parameters, and the behavioral pattern detection configuration based on the response information, and transmit a notification message to a designated terminal device via the communication interface when a behavioral pattern satisfies a notification condition.

19. The system according to claim 18, wherein the circuitry is configured to perform incremental learning on the classification model using newly accumulated affective-annotated text data to update model parameters and adapt the discrimination boundary to changing behavioral patterns.

20. A method comprising:
receiving, via a communication interface coupled to a packet-switched network, audio-derived text data and associated user identification data and time information from a terminal device;
executing affective state analysis processing on the text data using a natural language processing model to generate affective-annotated text data comprising at least one affective label and an affective score, and storing the affective-annotated text data in a storage device in association with the user identification data and the time information;
generating, from a set of stored affective-annotated text data, feature information representing behavioral tendency patterns, constructing a classification model that estimates a presence or degree of deviation in the behavioral tendency patterns based on the feature information, and executing behavioral pattern detection for each user using the classification model;
computing statistical information over time for the affective labels, affective scores, phrase occurrences, and classification model outputs, generating a prompt sentence based on the behavioral pattern detection results and the statistical information, executing inference processing using a generative neural network model with the prompt sentence as input, and receiving response information from the generative neural network model; and
updating at least one of parameters, processing content, and configuration of the affective state analysis processing and the behavioral pattern detection processing based on the response information.
